SEQUENCE LISTING (203 sequences)

SEQ ID NO: 1 - DNA, 3178 bp, Homo sapiens
gttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaaaaaaaaaaaaaaaaaaaaaaaaaaaaaaaaaaaaaaa

SEQ ID NO: 2 - DNA, 21 bp, Artificial Sequence
  Synthetic
ttagctcccacagatacctca

SEQ ID NO: 3 - DNA, 21 bp, artificial
  Synthetic
aaggttgttgtcctcatccct

SEQ ID NO: 4 - DNA, 21 bp, Artificial Sequence
  Synthetic
tatttgcaaggcccgatgtag

SEQ ID NO: 5 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 6 - protein, 286 aa, Homo sapiens
APQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 7 - DNA, 20 bp, Artificial Sequence
  Synthetic
ttccgccaccacctcctctt

SEQ ID NO: 8 - DNA, 19 bp, Artificial Sequence
  Synthetic
ttccgccaccacctcctct

SEQ ID NO: 9 - DNA, 14 bp, Artificial Sequence
  Synthetic
ttaggatagataga

SEQ ID NO: 10 - DNA, 15 bp, Artificial Sequence
  Synthetic
ggaattcctttccca

SEQ ID NO: 11 - DNA, 19 bp, Artificial Sequence
  Synthetic
gatgtacatgttatagttc

SEQ ID NO: 12 - DNA, 19 bp, Artificial Sequence
  Synthetic
gaagatgtacatgttatag

SEQ ID NO: 13 - DNA, 19 bp, Artificial Sequence
  Synthetic
caggattagctcccacaga

SEQ ID NO: 14 - DNA, 16 bp, Artificial Sequence
  Synthetic
ttagatcacaagatcc

SEQ ID NO: 15 - DNA, 16 bp, Artificial Sequence
  Synthetic
ttgtcgatatccttct

SEQ ID NO: 16 - DNA, 16 bp, Artificial Sequence
  Synthetic
gcttgatgccgaagca

SEQ ID NO: 17 - DNA, 16 bp, Artificial Sequence
  Synthetic
gccggcccactcatcc

SEQ ID NO: 18 - DNA, 16 bp, Artificial Sequence
  Synthetic
agggcgatgtacttgt

SEQ ID NO: 19 - DNA, 16 bp, Artificial Sequence
  Synthetic
cattgtccaggtcaca

SEQ ID NO: 20 - DNA, 16 bp, Artificial Sequence
  Synthetic
ggtctcgaaaaagcgg

SEQ ID NO: 21 - DNA, 16 bp, Artificial Sequence
  Synthetic
gtggtgcaatgctcca

SEQ ID NO: 22 - DNA, 16 bp, Artificial Sequence
  Synthetic
tggggatgaggggagc

SEQ ID NO: 23 - DNA, 16 bp, Artificial Sequence
  Synthetic
acgcagtggagccagc

SEQ ID NO: 24 - DNA, 16 bp, Artificial Sequence
  Synthetic
tcggtgtgggagaggt

SEQ ID NO: 25 - DNA, 16 bp, Artificial Sequence
  Synthetic
acccgtcaatggggtg

SEQ ID NO: 26 - DNA, 16 bp, Artificial Sequence
  Synthetic
ctggtccagctggccg

SEQ ID NO: 27 - DNA, 16 bp, Artificial Sequence
  Synthetic
aactgccagtgtacag

SEQ ID NO: 28 - DNA, 16 bp, Artificial Sequence
  Synthetic
ggaagatgtacatgtt

SEQ ID NO: 29 - DNA, 16 bp, Artificial Sequence
  Synthetic
atagttcttctcgaag

SEQ ID NO: 30 - DNA, 16 bp, Artificial Sequence
  Synthetic
tcccgggccagcagct

SEQ ID NO: 31 - DNA, 16 bp, Artificial Sequence
  Synthetic
ccacggggtggtctcc

SEQ ID NO: 32 - DNA, 16 bp, Artificial Sequence
  Synthetic
tgcctccaggcgcttc

SEQ ID NO: 33 - DNA, 16 bp, Artificial Sequence
  Synthetic
tcattctcatggatct

SEQ ID NO: 34 - DNA, 16 bp, Artificial Sequence
  Synthetic
tcttcacccgcagctt

SEQ ID NO: 35 - DNA, 16 bp, Artificial Sequence
  Synthetic
ctgcttctcagtcaga

SEQ ID NO: 36 - DNA, 16 bp, Artificial Sequence
  Synthetic
aggttgttgtcctcat

SEQ ID NO: 37 - DNA, 16 bp, Artificial Sequence
  Synthetic
ccctctcatacagggt

SEQ ID NO: 38 - DNA, 16 bp, Artificial Sequence
  Synthetic
gaccaggacgttcttg

SEQ ID NO: 39 - DNA, 16 bp, Artificial Sequence
  Synthetic
agccagtcccgcatgc

SEQ ID NO: 40 - DNA, 16 bp, Artificial Sequence
  Synthetic
gcagggggaattcggt

SEQ ID NO: 41 - DNA, 16 bp, Artificial Sequence
  Synthetic
cagctcagagtccagg

SEQ ID NO: 42 - DNA, 16 bp, Artificial Sequence
  Synthetic
caaggggggatgtatt

SEQ ID NO: 43 - DNA, 16 bp, Artificial Sequence
  Synthetic
tgcaaggcccgatgta

SEQ ID NO: 44 - DNA, 16 bp, Artificial Sequence
  Synthetic
gtccaggtggagcttg

SEQ ID NO: 45 - DNA, 16 bp, Artificial Sequence
  Synthetic
tggcccttcttggtgc

SEQ ID NO: 46 - DNA, 16 bp, Artificial Sequence
  Synthetic
cctccagggtgcactt

SEQ ID NO: 47 - DNA, 16 bp, Artificial Sequence
  Synthetic
tgtggcaaagaagtgg

SEQ ID NO: 48 - DNA, 16 bp, Artificial Sequence
  Synthetic
caggaagagtcgaagg

SEQ ID NO: 49 - DNA, 16 bp, Artificial Sequence
  Synthetic
tcttgttgtcattgct

SEQ ID NO: 50 - DNA, 16 bp, Artificial Sequence
  Synthetic
gcacaccttctcaaac

SEQ ID NO: 51 - DNA, 16 bp, Artificial Sequence
  Synthetic
tcgccaatgggggctg

SEQ ID NO: 52 - DNA, 16 bp, Artificial Sequence
  Synthetic
ggcagctggtggggtc

SEQ ID NO: 53 - DNA, 16 bp, Artificial Sequence
  Synthetic
ctggcacacgcacatg

SEQ ID NO: 54 - DNA, 16 bp, Artificial Sequence
  Synthetic
ggggtgttgttctcat

SEQ ID NO: 55 - DNA, 16 bp, Artificial Sequence
  Synthetic
ccagctcgcacacctt

SEQ ID NO: 56 - DNA, 16 bp, Artificial Sequence
  Synthetic
gccgtgtttgcagtgg

SEQ ID NO: 57 - DNA, 16 bp, Artificial Sequence
  Synthetic
tggttctggcagggat

SEQ ID NO: 58 - DNA, 16 bp, Artificial Sequence
  Synthetic
tttccgccaccacctc

SEQ ID NO: 59 - DNA, 16 bp, Artificial Sequence
  Synthetic
ctcttcggtttcctct

SEQ ID NO: 60 - DNA, 16 bp, Artificial Sequence
  Synthetic
gcaccatcatcaaatt

SEQ ID NO: 61 - DNA, 16 bp, Artificial Sequence
  Synthetic
ctcctacttccacctg

SEQ ID NO: 62 - DNA, 16 bp, Artificial Sequence
  Synthetic
gacaggattagctccc

SEQ ID NO: 63 - DNA, 16 bp, Artificial Sequence
  Synthetic
acagatacctcagtca

SEQ ID NO: 64 - DNA, 16 bp, Artificial Sequence
  Synthetic
cctctgccacagtttc

SEQ ID NO: 65 - DNA, 16 bp, Artificial Sequence
  Synthetic
ttccaccacctctgtc

SEQ ID NO: 66 - DNA, 16 bp, Artificial Sequence
  Synthetic
tcatcaggcagggctt

SEQ ID NO: 67 - DNA, 16 bp, Artificial Sequence
  Synthetic
cttgctgaggggctgc

SEQ ID NO: 68 - DNA, 16 bp, Artificial Sequence
  Synthetic
caaggccctcccggcc

SEQ ID NO: 69 - DNA, 16 bp, Artificial Sequence
  Synthetic
aggcaaaggagaaaga

SEQ ID NO: 70 - DNA, 16 bp, Artificial Sequence
  Synthetic
agatccaggccctcat

SEQ ID NO: 71 - DNA, 16 bp, Artificial Sequence
  Synthetic
atgagggcctggatct

SEQ ID NO: 72 - DNA, 16 bp, Artificial Sequence
  Synthetic
tctttctcctttgcct

SEQ ID NO: 73 - DNA, 16 bp, Artificial Sequence
  Synthetic
ggccgggagggccttg

SEQ ID NO: 74 - DNA, 16 bp, Artificial Sequence
  Synthetic
gcagcccctcagcaag

SEQ ID NO: 75 - DNA, 16 bp, Artificial Sequence
  Synthetic
aagccctgcctgatga

SEQ ID NO: 76 - DNA, 16 bp, Artificial Sequence
  Synthetic
gacagaggtggtggaa

SEQ ID NO: 77 - DNA, 16 bp, Artificial Sequence
  Synthetic
gaaactgtggcagagg

SEQ ID NO: 78 - DNA, 16 bp, Artificial Sequence
  Synthetic
tgactgaggtatctgt

SEQ ID NO: 79 - DNA, 16 bp, Artificial Sequence
  Synthetic
gggagctaatcctgtc

SEQ ID NO: 80 - DNA, 16 bp, Artificial Sequence
  Synthetic
caggtggaagtaggag

SEQ ID NO: 81 - DNA, 16 bp, Artificial Sequence
  Synthetic
aatttgatgatggtgc

SEQ ID NO: 82 - DNA, 16 bp, Artificial Sequence
  Synthetic
agaggaaaccgaagag

SEQ ID NO: 83 - DNA, 16 bp, Artificial Sequence
  Synthetic
gaggtggtggcggaaa

SEQ ID NO: 84 - DNA, 16 bp, Artificial Sequence
  Synthetic
atccctgccagaacca

SEQ ID NO: 85 - DNA, 16 bp, Artificial Sequence
  Synthetic
ccactgcaaacacggc

SEQ ID NO: 86 - DNA, 16 bp, Artificial Sequence
  Synthetic
aaggtgtgcgagctgg

SEQ ID NO: 87 - DNA, 16 bp, Artificial Sequence
  Synthetic
atgagaacaacacccc

SEQ ID NO: 88 - DNA, 16 bp, Artificial Sequence
  Synthetic
catgtgcgtgtgccag

SEQ ID NO: 89 - DNA, 16 bp, Artificial Sequence
  Synthetic
gaccccaccagctgcc

SEQ ID NO: 90 - DNA, 16 bp, Artificial Sequence
  Synthetic
cagcccccattggcga

SEQ ID NO: 91 - DNA, 16 bp, Artificial Sequence
  Synthetic
gtttgagaaggtgtgc

SEQ ID NO: 92 - DNA, 16 bp, Artificial Sequence
  Synthetic
agcaatgacaacaaga

SEQ ID NO: 93 - DNA, 16 bp, Artificial Sequence
  Synthetic
ccttcgactcttcctg

SEQ ID NO: 94 - DNA, 16 bp, Artificial Sequence
  Synthetic
ccacttctttgccaca

SEQ ID NO: 95 - DNA, 16 bp, Artificial Sequence
  Synthetic
aagtgcaccctggagg

SEQ ID NO: 96 - DNA, 16 bp, Artificial Sequence
  Synthetic
gcaccaagaagggcca

SEQ ID NO: 97 - DNA, 16 bp, Artificial Sequence
  Synthetic
caagctccacctggac

SEQ ID NO: 98 - DNA, 16 bp, Artificial Sequence
  Synthetic
tacatcgggccttgca

SEQ ID NO: 99 - DNA, 16 bp, Artificial Sequence
  Synthetic
aatacatccccccttg

SEQ ID NO: 100 - DNA, 16 bp, Artificial Sequence
  Synthetic
cctggactctgagctg

SEQ ID NO: 101 - DNA, 16 bp, Artificial Sequence
  Synthetic
accgaattccccctgc

SEQ ID NO: 102 - DNA, 16 bp, Artificial Sequence
  Synthetic
gcatgcgggactggct

SEQ ID NO: 103 - DNA, 16 bp, Artificial Sequence
  Synthetic
caagaacgtcctggtc

SEQ ID NO: 104 - DNA, 16 bp, Artificial Sequence
  Synthetic
accctgtatgagaggg

SEQ ID NO: 105 - DNA, 16 bp, Artificial Sequence
  Synthetic
atgaggacaacaacct

SEQ ID NO: 106 - DNA, 16 bp, Artificial Sequence
  Synthetic
tctgactgagaagcag

SEQ ID NO: 107 - DNA, 16 bp, Artificial Sequence
  Synthetic
aagctgcgggtgaaga

SEQ ID NO: 108 - DNA, 16 bp, Artificial Sequence
  Synthetic
agatccatgagaatga

SEQ ID NO: 109 - DNA, 16 bp, Artificial Sequence
  Synthetic
gaagcgcctggaggca

SEQ ID NO: 110 - DNA, 16 bp, Artificial Sequence
  Synthetic
ggagaccaccccgtgg

SEQ ID NO: 111 - DNA, 16 bp, Artificial Sequence
  Synthetic
agctgctggcccggga

SEQ ID NO: 112 - DNA, 16 bp, Artificial Sequence
  Synthetic
cttcgagaagaactat

SEQ ID NO: 113 - DNA, 16 bp, Artificial Sequence
  Synthetic
aacatgtacatcttcc

SEQ ID NO: 114 - DNA, 16 bp, Artificial Sequence
  Synthetic
ctgtacactggcagtt

SEQ ID NO: 115 - DNA, 16 bp, Artificial Sequence
  Synthetic
cggccagctggaccag

SEQ ID NO: 116 - DNA, 16 bp, Artificial Sequence
  Synthetic
caccccattgacgggt

SEQ ID NO: 117 - DNA, 16 bp, Artificial Sequence
  Synthetic
acctctcccacaccga

SEQ ID NO: 118 - DNA, 16 bp, Artificial Sequence
  Synthetic
gctggctccactgcgt

SEQ ID NO: 119 - DNA, 16 bp, Artificial Sequence
  Synthetic
gctcccctcatcccca

SEQ ID NO: 120 - DNA, 16 bp, Artificial Sequence
  Synthetic
tggagcattgcaccac

SEQ ID NO: 121 - DNA, 16 bp, Artificial Sequence
  Synthetic
ccgctttttcgagacc

SEQ ID NO: 122 - DNA, 16 bp, Artificial Sequence
  Synthetic
tgtgacctggacaatg

SEQ ID NO: 123 - DNA, 16 bp, Artificial Sequence
  Synthetic
acaagtacatcgccct

SEQ ID NO: 124 - DNA, 16 bp, Artificial Sequence
  Synthetic
ggatgagtgggccggc

SEQ ID NO: 125 - DNA, 16 bp, Artificial Sequence
  Synthetic
tgcttcggcatcaagc

SEQ ID NO: 126 - DNA, 16 bp, Artificial Sequence
  Synthetic
agaaggatatcgacaa

SEQ ID NO: 127 - DNA, 16 bp, Artificial Sequence
  Synthetic
ggatcttgtgatctaa

SEQ ID NO: 128 - DNA, 19 bp, Artificial Sequence
  Synthetic
gggtttagagacaggcaac

SEQ ID NO: 129 - DNA, 19 bp, Artificial Sequence
  Synthetic
ggaccgcgggaatgtggag

SEQ ID NO: 130 - DNA, 19 bp, Artificial Sequence
  Synthetic
gctctccgggcagtctgaa

SEQ ID NO: 131 - DNA, 19 bp, Artificial Sequence
  Synthetic
caggcggcaggcagagcgc

SEQ ID NO: 132 - DNA, 19 bp, Artificial Sequence
  Synthetic
accctcagtggcaggcagg

SEQ ID NO: 133 - DNA, 19 bp, Artificial Sequence
  Synthetic
ggccctcatggtgctggga

SEQ ID NO: 134 - DNA, 19 bp, Artificial Sequence
  Synthetic
aaaggagaaagaagatcca

SEQ ID NO: 135 - DNA, 19 bp, Artificial Sequence
  Synthetic
aaggccctcccggccaggc

SEQ ID NO: 136 - DNA, 19 bp, Artificial Sequence
  Synthetic
ttcttgctgaggggctgcc

SEQ ID NO: 137 - DNA, 19 bp, Artificial Sequence
  Synthetic
ctgtctcatcaggcagggc

SEQ ID NO: 138 - DNA, 19 bp, Artificial Sequence
  Synthetic
acagtttcttccaccacct

SEQ ID NO: 139 - DNA, 19 bp, Artificial Sequence
  Synthetic
tacctcagtcacctctgcc

SEQ ID NO: 140 - DNA, 19 bp, Artificial Sequence
  Synthetic
tctcctacttccacctgga

SEQ ID NO: 141 - DNA, 19 bp, Artificial Sequence
  Synthetic
ctctgcaccatcatcaaat

SEQ ID NO: 142 - DNA, 19 bp, Artificial Sequence
  Synthetic
ccacctcctcttcggtttc

SEQ ID NO: 143 - DNA, 19 bp, Artificial Sequence
  Synthetic
tggcagggattttccgcca

SEQ ID NO: 144 - DNA, 19 bp, Artificial Sequence
  Synthetic
gtgtttgcagtggtggttc

SEQ ID NO: 145 - DNA, 19 bp, Artificial Sequence
  Synthetic
ccagctcgcacaccttgcc

SEQ ID NO: 146 - DNA, 19 bp, Artificial Sequence
  Synthetic
atgggggtgttgttctcat

SEQ ID NO: 147 - DNA, 19 bp, Artificial Sequence
  Synthetic
ggggtcctggcacacgcac

SEQ ID NO: 148 - DNA, 19 bp, Artificial Sequence
  Synthetic
tgggggctgggcagctggt

SEQ ID NO: 149 - DNA, 19 bp, Artificial Sequence
  Synthetic
accttctcaaactcgccaa

SEQ ID NO: 150 - DNA, 19 bp, Artificial Sequence
  Synthetic
cttgttgtcattgctgcac

SEQ ID NO: 151 - DNA, 19 bp, Artificial Sequence
  Synthetic
ggcaggaagagtcgaaggt

SEQ ID NO: 152 - DNA, 19 bp, Artificial Sequence
  Synthetic
cactttgtggcaaagaagt

SEQ ID NO: 153 - DNA, 19 bp, Artificial Sequence
  Synthetic
cttggtgccctccagggtg

SEQ ID NO: 154 - DNA, 19 bp, Artificial Sequence
  Synthetic
ggtggagcttgtggccctt

SEQ ID NO: 155 - DNA, 19 bp, Artificial Sequence
  Synthetic
caaggcccgatgtagtcca

SEQ ID NO: 156 - DNA, 19 bp, Artificial Sequence
  Synthetic
gcaaggggggatgtatttg

SEQ ID NO: 157 - DNA, 19 bp, Artificial Sequence
  Synthetic
cggtcagctcagagtccag

SEQ ID NO: 158 - DNA, 19 bp, Artificial Sequence
  Synthetic
cgcatgcgcagggggaatt

SEQ ID NO: 159 - DNA, 19 bp, Artificial Sequence
  Synthetic
gacgttcttgagccagtcc

SEQ ID NO: 160 - DNA, 19 bp, Artificial Sequence
  Synthetic
tctcatacagggtgaccag

SEQ ID NO: 161 - DNA, 19 bp, Artificial Sequence
  Synthetic
aggttgttgtcctcatccc

SEQ ID NO: 162 - DNA, 19 bp, Artificial Sequence
  Synthetic
cttctgcttctcagtcaga

SEQ ID NO: 163 - DNA, 19 bp, Artificial Sequence
  Synthetic
ggatcttcttcacccgcag

SEQ ID NO: 164 - DNA, 19 bp, Artificial Sequence
  Synthetic
aggcgcttctcattctcat

SEQ ID NO: 165 - DNA, 19 bp, Artificial Sequence
  Synthetic
ggggtggtctcctgcctcc

SEQ ID NO: 166 - DNA, 19 bp, Artificial Sequence
  Synthetic
cccgggccagcagctccac

SEQ ID NO: 167 - DNA, 19 bp, Artificial Sequence
  Synthetic
ttatagttcttctcgaagt

SEQ ID NO: 168 - DNA, 19 bp, Artificial Sequence
  Synthetic
tacagggaagatgtacatg

SEQ ID NO: 169 - DNA, 19 bp, Artificial Sequence
  Synthetic
gctggccgaactgccagtg

SEQ ID NO: 170 - DNA, 19 bp, Artificial Sequence
  Synthetic
tcaatggggtgctggtcca

SEQ ID NO: 171 - DNA, 19 bp, Artificial Sequence
  Synthetic
ggtgtgggagaggtacccg

SEQ ID NO: 172 - DNA, 19 bp, Artificial Sequence
  Synthetic
cacgcagtggagccagctc

SEQ ID NO: 173 - DNA, 19 bp, Artificial Sequence
  Synthetic
tccatggggatgaggggag

SEQ ID NO: 174 - DNA, 19 bp, Artificial Sequence
  Synthetic
aaagcgggtggtgcaatgc

SEQ ID NO: 175 - DNA, 19 bp, Artificial Sequence
  Synthetic
ccaggtcacaggtctcgaa

SEQ ID NO: 176 - DNA, 19 bp, Artificial Sequence
  Synthetic
gcgatgtacttgtcattgt

SEQ ID NO: 177 - DNA, 19 bp, Artificial Sequence
  Synthetic
gccggcccactcatccagg

SEQ ID NO: 178 - DNA, 19 bp, Artificial Sequence
  Synthetic
tctgcttgatgccgaagca

SEQ ID NO: 179 - DNA, 19 bp, Artificial Sequence
  Synthetic
agatccttgtcgatatcct

SEQ ID NO: 180 - DNA, 21 bp, Artificial Sequence
  Synthetic
gttgttgtcctcatccctctc

SEQ ID NO: 181 - DNA, 20 bp, Artificial Sequence
  Synthetic
gttcttgagccagtcccgca

SEQ ID NO: 182 - DNA, 20 bp, Artificial Sequence
  Synthetic
tcttccaccacctctgtctc

SEQ ID NO: 183 - DNA, 19 bp, Artificial Sequence
  Synthetic
tcacctctgccacagtttc

SEQ ID NO: 184 - DNA, 19 bp, Artificial Sequence
  Synthetic
tacttccacctggacagga

SEQ ID NO: 185 - DNA, 19 bp, Artificial Sequence
  Synthetic
catcatcaaattctcctac

SEQ ID NO: 186 - DNA, 19 bp, Artificial Sequence
  Synthetic
cctctgcaccatcatcaaa

SEQ ID NO: 187 - DNA, 19 bp, Artificial Sequence
  Synthetic
cctctgcaccatcatcaaa

SEQ ID NO: 188 - DNA, 19 bp, Artificial Sequence
  Synthetic
gtcgaaggtcttgttgtca

SEQ ID NO: 189 - DNA, 19 bp, Artificial Sequence
  Synthetic
ggaagagtcgaaggtcttg

SEQ ID NO: 190 - DNA, 19 bp, Artificial Sequence
  Synthetic
cttctcagtcagaaggttg

SEQ ID NO: 191 - DNA, 19 bp, Artificial Sequence
  Synthetic
ctgcttctcagtcagaagg

SEQ ID NO: 192 - DNA, 19 bp, Artificial Sequence
  Synthetic
ctgcttctcagtcagaagg

SEQ ID NO: 193 - DNA, 19 bp, Artificial Sequence
  Synthetic
cttctcattctcatggatc

SEQ ID NO: 194 - DNA, 19 bp, Artificial Sequence
  Synthetic
tacagggaagatgtacatg

SEQ ID NO: 195 - DNA, 19 bp, Artificial Sequence
  Synthetic
cagggcgatgtacttgtca

SEQ ID NO: 196 - DNA, 19 bp, Artificial Sequence
  Synthetic
cttgtcgatatccttctgc

SEQ ID NO: 197 - DNA, 19 bp, Artificial Sequence
  Synthetic
ttagctcccacagatacct

SEQ ID NO: 198 - DNA, 19 bp, Artificial Sequence
  Synthetic
aaggttgttgtcctcatcc

SEQ ID NO: 199 - DNA, 19 bp, Artificial Sequence
  Synthetic
tatttgcaaggcccgatgt

SEQ ID NO: 200 - DNA, 16 bp, Artificial Sequence
  Synthetic
tcatcaggcagggctt

SEQ ID NO: 201 - DNA, 19 bp, Artificial Sequence
  Synthetic
actccatagacaccctcga

SEQ ID NO: 202 - DNA, 21 bp, Artificial Sequence
  Synthetic
gatgtagcccggaacgtttat

SEQ ID NO: 203 - DNA, 22 bp, Artificial Sequence
  Synthetic
tccctacctcctgttgttggaa

(19) United States
(12) Patent Application Publication
(10) Pub. No.: US 2012/0183538 A1
(43) Pub. Date: Jul. 19, 2012

(54) SPARC ANTISENSE COMPOSITIONS AND USES THEREOF

(75) Inventors: Vuong Trieu, Agoura Hills, CA (US); Larn Hwang, Arcadia, CA (US); Neil Desai, Los Angeles, CA (US)

(73) Assignee: ABRAXIS BIOSCIENCE, LLC, Los Angeles, CA (US)

(21) Appl. No.: 13/382,803

(22) PCT Filed: Jul. 9, 2010
(86) PCT No.: PCT/US10/41600

§ 371 Date: Apr. 2, 2012

Related U.S. Application Data
(60) Provisional application No. 61/224,431, filed on Jul. 9, 2009.

(51) Int. Cl. A61K 39/395; C07H 21/02; C07H 21/00; C07K 2/00; A61K 31/7088; A61K 31/712; A61K 38/14; A61K 38/50; A61K 33/24; A61P 35/00; A61P 19/10; A61P 35/02; A61P 17/02; A61P 19/02; A61P 9/10; A61P 17/06; A61P 1/16; A61P 13/12; A61P 9/12; C07H 21/04; B82Y 5/00

(52) U.S. Cl. 424/133.1; 536/24.5; 530/322; 514/44 A; 514/20.9; 514/19.2; 424/94.6; 424/649; 514/34; 514/19.3; 514/16.9; 514/18.6; 514/19.4; 514/19.5; 514/19.6; 514/16.6; 514/1.9; 514/15.7; 977/742; 977/915

(57) ABSTRACT

The invention provides SPARC antisense oligonucleotides and methods of their use in proliferative diseases such as cancer and hepatic fibrosis.

### CROSS REFERENCE TO RELATED APPLICATIONS

This application claims the benefit of U.S. Provisional Application No. 61/224,431, filed on Jul. 9, 2009, which is hereby incorporated by reference.

### BACKGROUND OF THE INVENTION

Secreted protein acidic and rich in cysteine (also known as osteonectin, BM40, or SPARC) (hereainfter “SPARC”), is a matrix-associated protein that elicits changes in cell shape, inhibits cell-cycle progression, and influences the synthesis of extracellular matrix (Bradshaw et al., Proc. Nat. Acad. Sci. USA 100: 6045-6050 (2003)). The murine SPARC gene was cloned in 1986 (Mason et al., EMBO J. 5: 1465-1472 (1986)) and a full-length human SPARC cDNA (SEQ ID NO: 1) was cloned and sequenced in 1987 (Swaroop et al., Genomics 2: 37-47 (1988)). SPARC expression is developmentally regulated, and is predominantly expressed in tissues undergoing remodeling during normal development or in response to injury. For example, high levels of SPARC protein are expressed in developing bones and teeth (see, e.g., Lane et al., FASEB J., 8, 163 173 (1994); Yan & Sage, J. Histochem. Cytochem. 47:1495-1505 (1999)).

SPARC is highly expressed in several aggressive cancers, while it is absent in the corresponding normal tissues (e.g., bladder, liver, ovary, kidney, gut, and breast) (Porter et al., J. Histochem. Cytochem., 43, 791 (1995)). In bladder cancer, for example, SPARC expression has been associated with advanced carcinoma. Invasive bladder tumors of stage T2 or greater have been shown to express higher levels of SPARC relative to bladder tumors of stage T1 (or less superficial tumors), and poorer prognosis (see, e.g., Yamanaka et al., J. Urology, 166, 2495 2499 (2001)). In meningiomas, SPARC expression has been associated only with invasive tumors (see, e.g., Rempel et al., Clincal Cancer Res., 5, 237 241 (1999)). SPARC expression also has been detected in 74.5% of in situ invasive breast carcinoma lesions (see, e.g., Bellahcene, et al., Am. J. Pathol., 146, 95 100 (1995)), and 54.2% of infiltrating ductal carcinoma of the breast (see, e.g., Kim et al., J. Korean Med. Sci., 13, 652 657 (1998)).

SPARC also plays a role in non-neoplastic proliferative diseases. Mesangial cell proliferation is a characteristic feature of many glomerular diseases and often precedes extracellular matrix expansion and glomerulosclerosis. In a model of experimental mesangioproliferative glomerulonephritis, SPARC mRNA was increased 5-fold by day 7 and was identified in the mesangium by in situ hybridization. SPARC has been implicated in the pathogenesis of atherosclerotic lesions. Plasma SPARC levels are elevated in patients with coronary artery disease (Masahiko et al., Obesity Res. 9:388-393 (2001)). The proliferation of vascular smooth muscle cells in the arterial intima plays a central role in the pathogenesis of atherosclerosis. SPARC is expressed in vascular smooth muscle cells and macrophages associated with atherosclerotic lesions. In addition, SPARC has been hypothesized to regulate the action of platelet-derived growth factor during vascular injury (Masahiko et al., Obesity Res. 9:388-393 (2001); Raines et al., Proc. Natl. Acad. Sci. USA 89:1281-1285 (1992)). A stimulatory effect of SPARC on endothelial PAI-1 production has been reported at the site of vascular injury (Hasselaar et al., J. Biol. Chem. 266:13178-13184 (1991)) and has been postulated to accelerate atherosclerosis (Masahiko et al., Obesity Res. 9:388-393 (2001)).

Recently, a genetic polymorphism in SPARC has been associated with susceptibility to scleroderma. Transforming growth factor beta1 (TGFbeta1) is a profibrotic cytokine that stimulates excessive collagen production in patients with scleroderma or other fibrotic diseases. Exogenous TGFbeta1 induced increased expression of both SPARC and type I collagen in cultured normal human fibroblasts, but this response was significantly blunted in the fibroblasts transfected with SPARC siRNA. While the SPARC siRNAs used inhibited of SPARC expression in cultured human fibroblasts, this effect required the transfection of the siRNAs in vitro (Zhou et al., Arthritis Rheum. 52(1):257-61 (2005)).

Advanced liver fibrosis can be induced in Sprague-Dawley rats by prolonged intraperitoneal administration of thioacetamide. Hepatic SPARC expression significantly increased during the development of liver fibrosis. A recombinant adenovirus carrying antisense SPARC (AdasSPARC) markedly attenuated the development of hepatic fibrosis in rats treated with thiocetamide, as assessed by decreased collagen deposition, lower hepatic content of hydroxyproline and less advanced morphometric stage of fibrosis. AdasSPARC treatment also reduced inflammatory activity (Knodell score) and suppressed transdifferentiation of hepatic stellate cell to the myofibroblasts like phenotype in vivo (Camino et al., J Gene Med. 10(9):993-1004 (2008)).

Accordingly, there remains a need to develop therapeutic approaches that can control the level of SPARC expression in various conditions and, in particular, reduce the level of SPARC expression. Further, while antisense approaches to the inhibition of SPARC expression have been developed, these have required combersome adenoviral vectors or direct cellular transfection and there remains a need for more efficient and potentially therapeutic antisense approaches to the inhibition of SPARC expression.

### BRIEF SUMMARY OF THE INVENTION

The invention provides a SPARC antisense oligonucleotide comprising one or more DNA, RNA, mixed DNA/RNA, Locked Nucleic Acid (LNA) or Peptide Nucleic Acid (PNA), that is complementary to SEQ ID NO:1 or SEQ ID NO:5.

The antisense oligonucleotide can comprise, for example, one or more of SEQ ID NOs: 2-4 and 7-13. The antisense composition can optionally comprise a pharmaceutically acceptable carrier.

In one aspect, the SPARC antisense oligonucleotide comprises 10 to 30 bases which are complementary to SEQ ID NO:1 or SEQ ID NO:5, wherein the administration of the SPARC antisense oligonucleotide to a cell reduces the level of SPARC protein in the cell. Preferably, administration of the composition reduces the level of RNA of SEQ ID NO: 1 or SPARC protein in that cell by at least 30%, preferably by at least 80%, more preferably by at least 100 fold, most by preferably at least 1,000 fold.

In another aspect, the invention provides a method for treating or preventing a disease in an animal comprising administering a therapeutically effective amount of a composition comprising a SPARC antisense oligonucleotide, wherein the SPARC antisense oligonucleotide comprises a nucleic acid of SEQ ID NOs: 2-4 and 7-13, or combinations thereof.

In another aspect, the invention provides locations in the SPARC cDNA which are useful for targeting with SPARC antisense oligonucleotide. In particular, antisense oligonucleotides of 12 to 19 bases are provided which are complementary to SEQ ID NO: 1 at one or more of nucleotides 212, 311, 312, 521, 825, 841, 969, 985, 1001, 1017 of SEQ ID NO: 1.

Suitable proliferative diseases include, without limitation, cancer, restenosis, fribrosis, osteoporosis, inflammatory diseases including arthritis or exaggerated wound healing.

Suitable animals for administering the SPARC antisense compositions provided by the invention and for the application of the methods provided by the invention to treat or prevent proliferative diseases include, without limitation, human patients.

### BRIEF DESCRIPTION OF THE DRAWINGS

FIG. 1 depicts the restriction map of the SPARC-GFP expression vector.

FIG. 2 depicts the fluorescence spectroscopy results for expression of the SPARC-GFP fusion construct in an exemplary stably transfected cell line, as compared to an un-transfected cell line.

FIG. 3 depicts GFP luminescence in SPARC-GFP expressing cells transfected with siRNA SPARC 1, siRNA SPARC-2, and siRNA SPARC-3 as compared to negative controls scramble miR and DharmaFect™ transfection reagent alone (Dharmacon, Lafayette, Colo.).

FIG. 4 depicts GFP luminescence in SPARC-GFP expressing cells transfected with LNA anti-SPARC-1, LNA anti-SPARC-2, and LNA anti-SPARC-3.

FIG. 5A depicts GFP luminescence in SPARC-GFP expressing cells transfected with si13347, si13346, si13345 (SEQ ID NOs: 201-203, respectively), PO-SPARC-1, PO-SPARC-1-1, and negative control, at 24 hours of incubation.

FIG. 5B depicts GFP luminescence in SPARC-GFP expressing cells transfected with si13347, si13346, si13345 (SEQ ID NOs: 201-203, respectively), PO-SPARC-1, PO-SPARC-1-1, and negative control, at 48 hours of incubation.

FIG. 6A depicts GFP luminescence at 24 hours incubation in SPARC-GFP expressing cells transfected with LNA PO-SPARC-1, LNA PO-SPARC 1-1, LNA anti-SPARC 2, anti SP-53, siRNA SPARC-2, and DharmaFect1™ transfection reagent alone (Dharmacon, Lafayette, Colo.).

FIG. 6B depicts GFP luminescence at 48 hours incubation in SPARC-GFP expressing cells transfected with LNA PO-SPARC-1, LNA PO-SPARC 1-1, LNA anti-SPARC 2, anti SP-53, siRNA SPARC-2, and DharmaFect1™ transfection reagent alone (Dharmacon, Lafayette, Colo.).

FIG. 6C depicts GFP luminescence at 72 hours incubation in SPARC-GFP expressing cells transfected with LNA PO-SPARC-1, LNA PO-SPARC 1-1, LNA anti-SPARC 2, anti SP-53, siRNA SPARC-2, and DharmaFect1™ transfection reagent alone (Dharmacon, Lafayette, Colo.).

FIG. 7A depicts cytotoxicity at 48 hours incubation in SPARC-GFP expressing cells transfected with LNA PO-SPARC-1, LNA PO-SPARC 1-1, LNA anti-SPARC 2, anti SP-53, siRNA SPARC-2, and DharmaFect1™ transfection reagent alone (Dharmacon, Lafayette, Colo.).

FIG. 7B depicts cytotoxicity at 72 hours incubation in SPARC-GFP expressing cells transfected with LNA PO-SPARC-1, LNA PO-SPARC 1-1, LNA anti-SPARC 2, anti SP-53, siRNA SPARC-2, and DharmaFect1™ transfection reagent alone (Dharmacon, Lafayette, Colo.).

FIG. 8A depicts GFP luminescence at 48 hours incubation in SPARC-GFP expressing cells transfected with AS-SPARC-12 (SEQ ID NO:11), AS-SPARC-13 (SEQ ID NO:12), AS-SPARC-32 (SEQ ID NO:13), siRNA-SPARC-2 (SEQ ID NO: 202) and a negative control (DharmaFect1™ transfection agent (Dharmacon, Lafayette, Colo.)).

FIG. 8B depicts cytotoxicity at 48 hours incubation in SPARC-GFP expressing cells transfected with AS-SPARC-12 (SEQ ID NO:11), AS-SPARC-13 (SEQ ID NO:12), AS-SPARC-32 (SEQ ID NO:13), siRNA-SPARC-2 (SEQ ID NO: 202) and a negative control (DharmaFect1™ transfection agent (Dharmacon, Lafayette, Colo.)).

FIG. 9 depicts the human SPARC cDNA sequence (SEQ ID NO: 1)

FIG. 10 depicts the human SPARC full length/unprocessed (SEQ ID NO: 5) and mature/processed (SEQ ID NO: 6) amino acid sequences.

FIG. 11 conceptually depicts hot spots in the SPARC cDNA for targeting with SPARC antisense oligonucleotides.

### DETAILED DESCRIPTION OF THE INVENTION

### I. Definitions

As used herein the term “SPARC protein” refers to a polypeptide of with an identical sequence to either the unprocessed (SEQ ID NO: 5) or mature SPARC polypeptide (SEQ ID NO: 6) or a natural splice variant generated from SEQ ID NO: 1 or a polypeptide of substantially the identical sequence to either SEQ ID NO: 5 or 6 and which substantially retains the function of the mature SPARC polypeptide. By “a substantially the identical sequence” it is meant that the sequence is at least 80% identical, preferably at least 85% identical, more preferably at least 90% identical, even more preferably at least 95% identical, and most preferably at least 99% identical to either SEQ ID NOS: 5 or 6.

By “substantially retains the function of the mature SPARC” it is meant that the polypeptide has one or more of the biological/biochemical activities of SPARC known to those of ordinary skill, particularly activities that effect (maintain, support, induce, cause, diminish, prevent or inhibit) a disease state, including, e.g., influencing angiogenesis, cell shape, cell motility, cell adhesion, apoptosis, cellular proliferation or the composition of the extracellular matrix. Said polypeptides encompassed by the term “SPARC protein” also include polypeptides which have about 50 amino acids, preferably about 40 amino acids, more preferably about 30 amino acids, even more preferably about 20 amino acids, and most preferably about 10 amino acids added to the amino and/or carboxyl termini of a sequence that is identical to or substantially identical to SEQ ID NOS: 5 or 6.

A selection of sequences used herein are provided in Table 1.

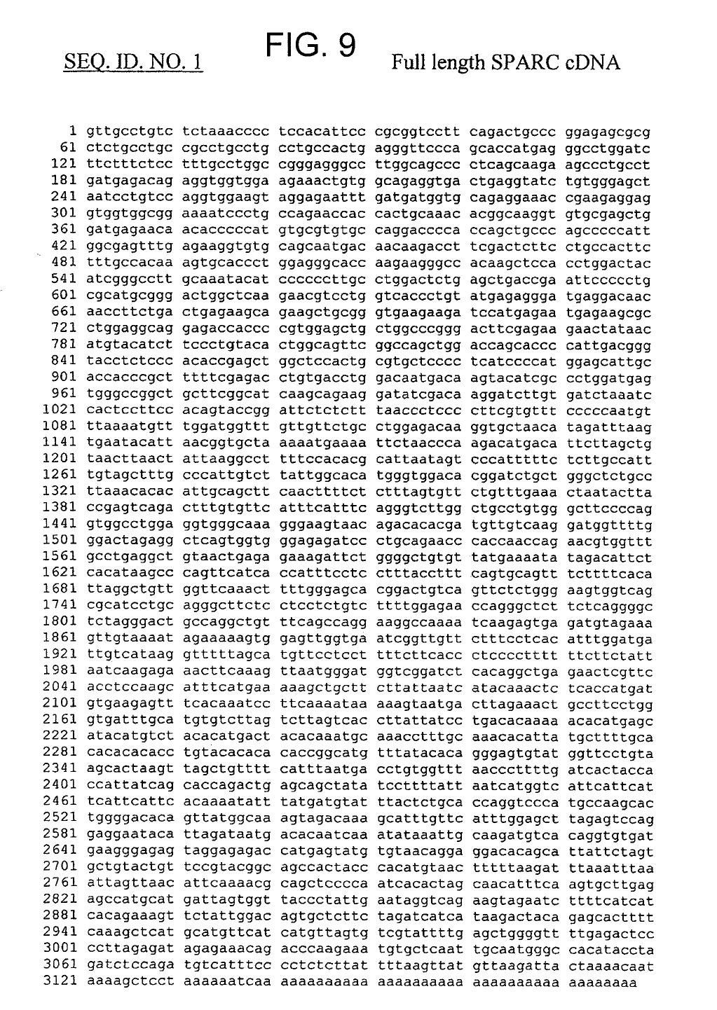

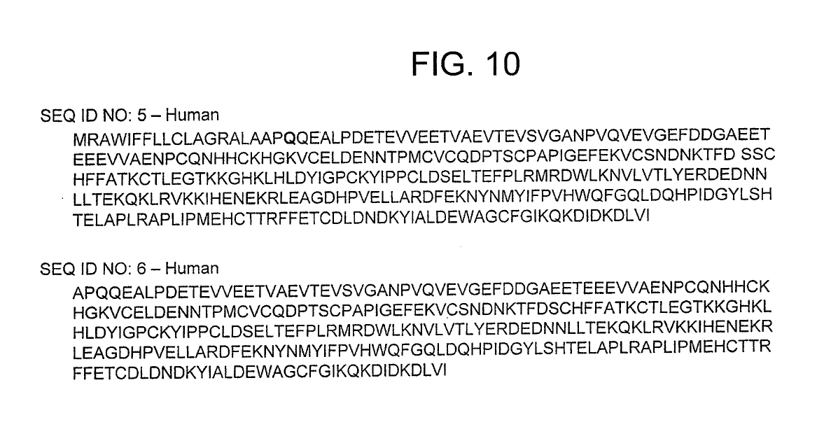

TABLE 1

| SEQ | | |
|---|---|---|
| ID | | |
| NO: | Name | Sequence |
| 1 | Full length | See FIG. 9 |
| | SPARC RNA | |
| 2 | LNA anti-SPARC | T*t*A*g*C*t*C*c*C*a*C*a*G*a*T*a*C*c*T*c*a |
| | 1 | |
| 3 | LNA anti-SPARC | A*a*G*g*T*t*G*t*T*g*T*c*C*t*C*a*T*c*C*c*t |
| | 2 | |
| 4 | LNA anti-SPARC | T*a*T*t*T*g*C*a*A*g*G*c*C*c*G*a*T*g* T*a*g |
| | 3 | |
| 5 | Unprocessed | See FIG. 10 |
| | SPARC protein | |
| 6 | Processed | See FIG. 10 |
| | SPARC protein | |
| 7 | PO-SPARC-1 | t*t*c*c*g*c*c*a*c*c*a*c*c*t*c*c*t*c*t*t |
| 8 | PO-SPARC-1-1 | TTCCG*c*c*a*c*c*a*c*c*TCCTCT |
| 9 | random 1 | ttaggatagataga |
| 10 | random | ggaattcctttccca |
| | 2 | |
| 11 | AS-SPARC-12 | +G* + A* + T* + G* + T*A*C*A*T*G*T*T*A*T* + A* + G* + T |
| | | * + T* + C |
| 12 | AS-SPARC-13 | +G* + A* + A* + G* + A*T*G*T*A*C*A*T*G*T* + T* + A* + T |
| | | * + A* + G |
| 13 | AS-SPARC-32 | +C* + A* + G* + G* + A*T*T*A*G*C*T*C*C*C* + A* + C* + A |
| | | * + G* + A |

*indicates PS
+ indicates LNA
uppercase letter (e.g., C, G, A, T), indicates LNA (except in SEQ ID NOS: 11-13, where it has no significance)
lowercase letter (e.g., c, g, a, t), indicates no modification

As used herein the term “SPARC RNA” refers to an RNA molecule comprising the coding sequence of a SPARC protein.

As used herein the term “nucleic acid” or “oligonucleotide” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Other such modifications are well known to those of skill in the art. Thus, the term nucleic acid also encompasses nucleic acids with substitutions or modifications, such as in the bases and/or sugars.

The term “base” encompasses any of the known base analogs of DNA and RNA. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

In addition, as used herein, the term “nucleic acid” includes peptide nucleic acids. Locked nucleic acids (LNA) are a class of nucleic acid analogues in which the ribose ring is “locked” by a methylene bridge connecting the 2′-O atom and the 4′-C atom. LNA nucleosides contain the common nucleobases (T, C, G, A, U and mC) and are able to form base pairs according to standard Watson-Crick base pairing rules. However, by “locking” the molecule with the methylene bridge the LNA is constrained in the ideal conformation for Watson-Crick binding. When incorporated into a DNA oligonucleotide, LNA therefore makes the pairing with a complementary nucleotide strand more rapid and increases the stability of the resulting duplex.

Further, as used herein the term “isolating RNA” includes preparing RNA in a histologic section for in situ hybridization.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

A “LNA/DNA mixmer” or “mixmer” is used to refer to a nucleic acid that contains at least one LNA unit and at least one RNA or DNA unit (e.g., a naturally-occurring RNA or DNA unit).

A “gapmer” is based on a central stretch of 4-12 base DNA (gap) typically flanked by 1 to 6 residues of 2′-O modified nucleotides (beta-D-oxy-LNA in our case, flanks) which are able to act via an RNaseH mediated mechanism to reduce the target sequence's level.

A “headmer” is defined by a contiguous stretch of beta-D-oxy-LNA or LNA derivatives at the 5′-end followed by a contiguous stretch of DNA or modified monomers recognizable and cleavable by the RNaseH towards the 3′-end, and a “tailmer” is defined by a contiguous stretch of DNA or modified monomers recognizable and cleavable by the RNaseH at the 5′-end followed by a contiguous stretch of .beta-D-oxy-LNA or LNA derivatives towards the 3′-end. Suitably, in one such “gapmer” embodiment, said subsequence comprises a stretch of 4 nucleotide analogues, such as LNA nucleotide analogues, as defined herein, followed by a stretch of 8 nucleotides, which is followed by a stretch of 4 nucleotide analogues, such as LNA nucleotide analogues as defined herein, optionally with a single nucleotide at the 3′ end.

In one further “gapmer” embodiment, said subsequence comprises a stretch of 3 nucleotide analogues, such as LNA nucleotide analogues, as defined herein, followed by a stretch of 9 nucleotides, which is followed by a stretch of 3 nucleotide analogues, such as LNA nucleotide analogues as defined herein, optionally with a single nucleotide at the 3′ end. Such a design has surprisingly been found to be very effective.

In one further “gapmer” embodiment, said subsequence comprises a stretch of 4 nucleotide analogues, such as LNA nucleotide analogues, as defined herein, followed by a stretch of 8 nucleotides, which is followed by a stretch of 3 nucleotide analogues, such as LNA nucleotide analogues as defined herein, optionally with a single nucleotide at the 3′ end.

The term “conjugate” or “tag” refers to a chemical moiety, either a nucleotide, oligonucleotide, polynucleotide or an amino acid, peptide or protein or other chemical, that when added to another sequence, provides additional utility or confers useful properties, particularly in the delivery, trafficking, detection or isolation of that sequence. Preferably, the conjugate is cholesterol added to the 3′ end of the MRE-concealing LNA, which confers the ability of the LNA of the invention to be cell permeable. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Numerous other tag moieties are known to, and can be envisioned by, the skilled artisan, and are contemplated to be within the scope of this definition.

As used herein, the term “tumor” refers to any neoplastic growth, proliferation or cell mass whether benign or malignant (cancerous), whether a primary site lesion or metastases.

As used herein, the term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Over 200 different types of cancers are known, and every organ and tissue of the body can be affected. Specific examples of cancers that do not limit the definition of cancer can include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. Alternatively, a cancer can be multicentric or of unknown primary site (CUPS).

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

In some embodiments, the term “therapeutically effective” refers to a result which substantially decreases the level or expression of, including for example, an about 20% reduction, preferrably an about 25% reduction, more preferrably an about 30% reduction, even more preferrably an about 33% reduction, even more preferrably an about 50% reduction, even more preferrably an about 67% reduction, even more preferrably an about 80% reduction, even more preferrably an about 90% reduction, even more preferrably an about 95% reduction, even more preferrably an about 99% reduction, even more preferrably an about 50 fold reduction, even more preferrably an about 100 fold reduction, even more preferrably an about 1,000 fold reduction, even more preferrably an about 10,000 fold reduction, and most preferable complete silencing.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent” according to the present invention, also includes a radiation therapy agent or a “chemotherapuetic agent.”

As used herein, the term “diagnostic agent” refers to any chemical used in the imaging of diseased tissue, such as, e.g., a tumor.

As used herein, the teem “chemotherapuetic agent” refers to an agent with activity against cancer, neoplastic, and/or proliferative diseases, or that has ability to kill cancerous cells directly.

As used herein, “pharmaceutical formulations” include formulations for human and veterinary use with no significant adverse toxicological effect. “Pharmaceutically acceptable formulation” as used herein refers to a composition or formulation that allows for the effective distribution of the nucleic acid molecules of the instant invention in the physical location most suitable for their desired activity.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

As used herein “therapeutically effective amount” refers to an amount of a composition that relieves (to some extent, as judged by a skilled medical practitioner) one or more symptoms of the disease or condition in a mammal. Additionally, “therapeutically effective amount” refers to an amount of a composition that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But, it is within the skill of an ordinarily skilled clinician upon the appreciation of the disclosure set forth herein.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent”, according to the present invention, also includes a radiation therapy agent or a “chemotherapeutic agent.”

As used herein, the term “diagnostic agent” refers to any chemical used in the imaging of diseased tissue, such as, e.g., a tumor.

As used herein, the term “chemotherapeutic agent” refers to an agent with activity against cancer, neoplastic, and/or proliferative diseases.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

As used herein the term “alternative therapeutic regimen” or “alternative therapy” (not a first line chemotherapeutic regimen as described above) may include for example, receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rouse sarcoma virus transforming oncogene (SRC) inhibitors, histonedeacetylase inhibitors, small hypoxia-inducible factor inhibitors, hedgehog inhibitors, and TGF-β signalling inhibitors. Furthermore, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Methods of use of chemotherapeutic agents and other agents used in alternative therapeutic regimens in combination therapies, including dosing and administration regimens, will also be known to a one skilled in the art.

The terms “co-administration” and “combination therapy” refer to administering to a subject two or more therapeutically active agents. The agents can be contained in a single pharmaceutical composition and be administered at the same time, or the agents can be contained in separate formulation and administered serially to a subject. So long as the two agents can be detected in the subject at the same time, the two agents are said to be co-administered.

### II. Oligonucleotides

The invention provides SPARC antisense oligonucleotides comprising one or more DNA, RNA, LNA or PNA oligonucleotides complementary to SEQ ID NO: 1 or 5.

In preferred embodiments, the oligonucleotide can have a sequence selected from SEQ ID NOs: 2-4 and 7-13. In other embodiments, the sequence is at least 80% identical, at least 90% identical, at least 95% identical, or at least 99% identical to any of SEQ ID NOs: 2-4 and 7-13. In any contemplated embodiment, however, the oligonucleotide is capable of specifically hybridizing to the sequence of SEQ ID NO: 1 or SEQ ID NO: 5.

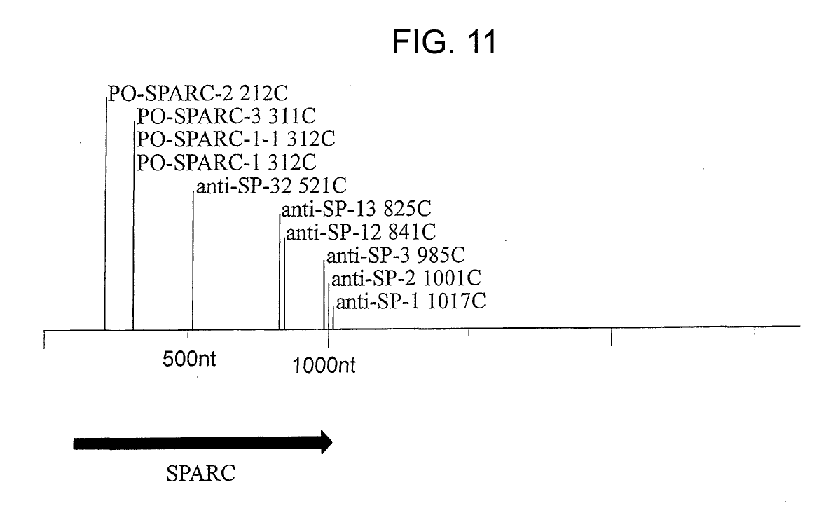

The invention further provides locations in the SPARC cDNA which are useful for targeting with SPARC antisense oligonucleotide. In particular, antisense oligonucleotides of 12 to 19 bases are provided which are complementary to SEQ ID NO: 1 at one or more of nucleotides 212, 311, 312, 521, 825, 841, 969, 985, 1001, 1017 of SEQ ID NO: 1, as shown conceptually at FIG. 11. That is, antisense oligonucleotides are provided which are complementary to one or more of the aforementioned identified nucleotides of SEQ ID NO: 1 as well as additional consecutive nucleotides located on one or both sides of the identified nucleotide(s). One of ordinary skill in the art will understand that degenerate or modified nucleotides are further contemplated but must also be capable of specifically hybridizing to the sequence of SEQ ID NO: 1. For example, an oligonucleotide could differ from the complementary sequence by three nucleotides, two nucleotides, or preferably one nucleotide, although oligonucleotides having the complementary sequence itself are most preferred.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a RNA molecule, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

Suitable oligonucleotides can be unmodified or chemically modified single-stranded oligonucleotides. Suitable oligonucleotides are from about 10 to about 30 bases in length, preferably from about 12 to about 25 bases in length. Most preferably, oligonucleotides are about 12 to about 19 bases in length.

SPARC antisense oligonucleotides in accordance with the invention include compositions where one or more DNA, RNA, LNA or PNA molecules comprises any one or more of SEQ ID NOS: 2-4 and 7-13. In preferred embodiments the invention provides SPARC antisense oligonucleotides comprising SEQ ID NOS: 3 and/or 8. SPARC antisense oligonucleotides in accordance with the invention can further comprising a pharmaceutically-acceptable carrier.

The SPARC antisense oligonucleotides provided by the invention can include one or more of a gapmer, mixmer, 2′-MOE, phosphorothioate boranophosphate, 2′-O-methyl, 2′-fluoro, terminal inverted-dT bases, PEG, 2′ tBDMS, 2′-TOM, t′-ACE or combinations thereof. The SPARC antisense oligonucleotides provided by the invention include those where at least one of said one or more DNA, RNA, LNA or PNA oligonucleotides is modified by the addition of any one of cholesterol, bis-cholesterol, PEG, PEG-ylated carbon nanotube, poly-L-lysine, cyclodextran, polyethylenimine polymer or peptide moieties. Further, the SPARC antisense oligonucleotides provided by the invention include those in which each of said one or more DNA, RNA, LNA or PNA oligonucleotides is modified by the addition of any one of cholesterol, bis-cholesterol, PEG, PEG-ylated carbon nanotube, poly-L-lysine, cyclodextran, polyethylenimine polymer or peptide moieties. Further, oligonucleotides in accordance with the invention can be modified by any polymeric species including synthetic or naturally occurring polymers or proteins.

Suitable oligonucleotides for use in accordance with the invention can be composed of naturally occurring nucleobases, sugars and internucleoside (backbone) linkages as well as oligonucleotides having non-naturally-occurring portions which function similarly or with specific improved functions. Fully or partly modified or substituted oligonucleotides are often preferred over native forms because of several desirable properties of such oligonucleotides, for instance, the ability to penetrate a cell membrane, good resistance to extra- and intracellular nucleases, high affinity and specificity for the nucleic acid target.

Preferably, the oligomeric compound, such as an antisense oligonucleotide, according to the invention comprises at least one Locked Nucleic Acid (LNA) unit, such as 3, 4, 5, 6, 7, 8, 9, or 10 Locked Nucleic Acid (LNA) units, preferably between 4 to 9 LNA units, such as 6-9 LNA units, most preferably 6, 7 or 8 LNA units. Preferably the LNA units comprise at least one beta-D-oxy-LNA unit(s) such as 4, 5, 6, 7, 8, 9, or 10 beta-D-oxy-LNA units. All the LNA units can, e.g., be beta-D-oxy-LNA units, although it is considered that the oligomeric compounds, such as the antisense oligonucleotide, may comprise more than one type of LNA unit. Suitably, the oligomeric compound may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

Embodiments of the invention can comprise nucleotide analogues, such as LNA nucleotide analogues, the subsequence typically may comprise a stretch of 2-6 nucleotide analogues, such as LNA nucleotide analogues, as defined herein, followed by a stretch of 4-12 nucleotides, which is followed by a stretch of 2-6 nucleotide analogues, such as LNA nucleotide analogues, as defined herein. One suitable embodiment, the oligonucleotides of the instant invention comprise modified bases such that the oligonucleotides retain their ability to bind other nucleic acid sequences, but are unable to associate significantly with proteins such as the RNA degradation machinery. LNAs confer increased affinity to the target, and are a preferred embodiment within the scope of the invention. For increased nuclease resistance and/or binding affinity to the target, the oligonucleotide agents featured in the invention can also include 2′-O-methyl, 2′-fluorine, 2′-O-methoxyethyl, 2′-β-aminopropyl, 2′-amino, and/or phosphorothioate linkages and the like. Inclusion of LNAs, ethylene nucleic acids (ENAS), e.g., 2′-4′-ethylene-bridged nucleic acids, and certain nucleobase modifications such as 2-amino-A, 2-thio (e.g., 2-thio-U), G-clamp modifications, can also increase binding affinity to the target.

Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA.

While deoxyribonucleotide phosphodiester oligonucleotides are suitable for use in accordance with the invention, they are not preferred. Methylphosphonate oligonucleotides are noncharged oligomers, in which a nonbridging oxygen atom is replaced by a methyl group at each phosphorus in the oligonucleotide chain. The phosphorothioates in the phosphorothioate diastereomer have improved nuclease stability. A preferred embodiment involves the replacement of the hydrogen at the 2′-position of ribose by an O-alkyl group, most frequently methyl. These oligonucleotides form high melting heteroduplexes with targeted mRNA and induce an antisense effect by a non-RNase H-dependent mechanism.

Suitable oligonucleotides also include embodiments that do not possess the natural phosphate-ribose backbone. Peptide Nucleic Acids (PNAs) are nucleic acid analogues that contain an uncharged, flexible, polyamide backbone comprised of repeating N-(2-aminoethyl) glycine units to which the nucleobases are attached via methylene carbonyl linkers. These oligomers can form very stable duplexes or triplexes with nucleic acids: single or double-strand DNA or RNA. The property of high-affinity nucleic acid binding can be explained by the lack of electrostatic repulsion because of the absence of negative charges on the PNA oligomers. Because PNAs are not substrates for the RNase H or other RNases, the antisense mechanism of PNAs depends on steric hindrance. PNAs can also bind to DNA and inhibit RNA polymerase initiation and elongation, as well as the binding and action of transcription factors, such as nuclear factor KB. PNAs can also bind mRNA and inhibit splicing or translation initiation and elongation.

Phosphorodiamidate morpholino oligomers, in which the deoxyribose moiety is replaced by a morpholine ring and the charged phosphodiester intersubunit linkage is replaced by an uncharged phosphorodiamidate linkage, are also suitable for use in accordance with the invention. These oligonucleotides are very stable in biological systems and exhibit efficient antisense activity in cell-free translation systems and in a few cultured animal cell lines.

Another example of a suitable type of oligonucleotide is the N3′→P5′ PN, which result from the replacement of the oxygen at the 3′ position on ribose by an amine group. These oligonucleotides can, relative to their isosequential phosphodiester counterparts, foam very stable complexes with RNA and single- or double-stranded DNA. Specificity, as well as efficacy, can be increased by using a chimeric oligonucleotide, in which the RNase H-competent segment, usually a phosphorothioate moiety, is bounded on one or both termini by a higher-affinity region of modified RNA, e.g., a 2′O-alkyloligoribonucleotides. This substitution not only increases the affinity of the oligonucleotide for its target but reduces the cleavage of nontargeted mRNAs by RNase H.

In preferred embodiments, the administration of the SPARC antisense composition to a cell reduces the level of RNA of SEQ ID NO: 1 or SPARC protein in that cell by at least 25%, at least 30%, at least 80%, at least 100 fold, or most by preferably at least 1,000 fold.

### III. Pharmaceutical Compositions

In some embodiments, the SPARC antisense compositions of the present invention can further comprise a pharmaceutically acceptable carrier.

The compositions of the present invention can further comprise an active agent. In some embodiments, the active agent is a pharmaceutically active therapeutic agent directly able to exert its pharmacological effect. In other embodiments, the active agent is a diagnostic agent. In preferred embodiments, the active agent is a diagnostic or therapeutic active agent conjugated to a SPARC antisense oligonucleotide. It will be understood that some active agents are useful as both diagnostic and therapeutic agents, and therefore such twins are not mutually exclusive.

The active agent can be any suitable therapeutic agent or diagnostic agent, such as a chemotherapeutic or anticancer agent. Suitable chemotherapeutic agents or other anticancer agents for use in accordance with the invention include but, are not limited to, tyrosine kinase inhibitors (genistein), biologically active agents (TNF, or tTF), radionuclides (131I, 90Y, 111In, 211At, 32P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, and transplatinum.

Other suitable chemotherapeutic agents for use in accordance with invention include, without limitation, antimetabolites (e.g., asparaginase), antimitotics (e.g., vinca alkaloids), DNA damaging agents (e.g., cisplatin), proapoptotics (agents which induce programmed-cell-death or apoptosis) (e.g, epipodophylotoxins), differentiation inducing agents (e.g., retinoids), antibiotics (e.g., bleomycin), and hormones (e.g., tamoxifen, diethylstilbestrol). Further, suitable chemotherapeutic agents for use in accordance with the invention include antiangiogenesis agents (angiogenesis inhibitors) such as, e.g., INF-alpha, fumagillin, angiostatin, endostatin, thalidomide, and the like.

Preferred chemotherapeutic agents include docetaxel, paclitaxel, and combinations thereof “Combinations thereof” refers to both the administration of dosage forms including more than one drug, for example, docetaxel and paclitaxel, as well as the sequential but, temporally distinct, administration of docetaxel and paclitaxel (e.g., the use of docetaxel in one cycle and paclitaxel in the next). Particularly preferred chemotherapeutic agents comprise particles of protein-bound drug, including but not limited to, wherein the protein making up the protein-bound drug particles comprises albumin including wherein more than 50% of the chemotherapeutic agent is in nanoparticle form. Most preferably the chemotherapeutic agent comprises particles of albumin-bound paclitaxel, such as, e.g., Abraxane®. Such albumin-bound paclitaxel formulations can be used in accordance with the invention where the paclitaxel dose administered is from about 30 mg/mL to about 1000 mg/mL with a dosing cycle of about 3 weeks (i.e., administration of the paclitaxel dose once every about three weeks). Further, it is desirable that the paclitaxel dose administered is from about 50 mg/mL to about 800 mg/mL, preferably from about 80 mg/mL to about 700 mg/mL, and most preferably from about 250 mg/mL to about 300 mg/mL with a dosing cycle of about 3 weeks.

Other therapeutic agents also include, without limitation, biologically active polypeptides, antibodies and fragments thereof, lectins, and toxins (such as ricin A), or radionuclides. Suitable antibodies for use as active agents in accordance with the invention include, without limitation, conjugated (coupled) or unconjugated (uncoupled) antibodies, monoclonal or polyclonal antibodies, humanized or unhumanized antibodies, as well as Fab′, Fab, or Fab2 fragments, single chain antibodies and the like. Contemplated antibodies or antibody fragments can be Fc fragments of IgG, IgA, IgD, IgE, or IgM. In various preferred embodiments, the active agent is a single chain antibody, a Fab fragment, diabody, and the like. In more preferred embodiments, the antibody or antibody fragment mediates complement activation, cell mediated cytotoxicity, and/or opsonization.

In addition, the pharmaceutically active agent can be an siRNA. In preferred embodiments, the siRNA molecule inhibits expression of an gene associated with tumors such as, for example, c-Sis and other growth factors, EGFR, PDGFR, VEGFR, HER2, other receptor tyrosine kinases, Src-family genes, Syk-ZAP-70 family genes, BTK family genes, other cytoplasmic tyrosine kinases, Raf kinase, cyclin dependent kinases, other cytoplasmic serine/threonine kinases, Ras protein and other regulatory GTPases.

SPARC antisense oligonucleotide can also be conjugated to polyethylene glycol (PEG). PEG conjugation can increase the circulating half-life of a protein, reduce the protein's immunogenicity and antigenicity, and improve the bioactivity. Any suitable method of conjugation can be used, including but not limited to, e.g., reacting methoxy-PEG with a SPARC antisense oligonucleotide available amino groups or other reactive sites such as, e.g., histidines or cysteines. In addition, recombinant DNA approaches can be used to add amino acids with PEG-reactive groups to the inventive SPARC antisense oligonucleotide. PEG can be processed prior to reacting it with a SPARC antisense oligonucleotide, e.g., linker groups can be added to the PEG. Further, releasable and hybrid PEG-ylation strategies can be used in accordance with the invention, such as, e.g., the PEG-ylation of a SPARC antisense oligonucleotide such that the PEG molecules added to certain sites in the SPARC antisense oligonucleotide are released in vivo. Such PEG conjugation methods are known in the art (See, e.g., Greenwald et al., Adv. Drug Delivery Rev. 55:217-250 (2003)).

Contemplated SPARC antisense oligonucleotides and conjugates thereof can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present inventions are generally provided in a formulation with a carrier, such as a pharmaceutically acceptable carrier. Typically, the carrier will be liquid, but also can be solid, or a combination of liquid and solid components. The carrier desirably is a physiologically acceptable (e.g., a pharmaceutically or pharmacologically acceptable) carrier (e.g., excipient or diluent). Physiologically acceptable carriers are well known and are readily available. Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized. Preferred physiologically acceptable carrier media are water, buffered water, normal saline, 0.4% saline, 0.3% glycine, hyaluronic acid and the like. The choice of carrier will be determined, at least in part, by the location of the target tissue and/or cells, and the particular method used to administer the composition.

The composition can be formulated for administration by a route including intravenous, intraarterial, intramuscular, intraperitoneal, intrathecal, epidural, topical, percutaneous, subcutaneous, transmucosal (including, for example, pulmonary), intranasal, rectal, vaginal, or oral. The composition also can comprise additional components such as diluents, adjuvants, excipients, preservatives, and pH adjusting agents, and the like.

Formulations suitable for injectable administration include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, lyoprotectants, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, or tablets.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In all cases, the formulation must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

In preferred embodiments, the active ingredients can be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the SPARC antisense oligonucleotide s can be prepared by such methods as described in Rezler et al., J. Am. Chem. Soc. 129(16): 4961-72 (2007); Samad et al., Curr. Drug Deliv. 4(4): 297-305 (2007); and U.S. Pat. Nos. 4,485,045 and 4,544,545. Liposomes with enhanced circulation time are disclosed in U.S. Pat. No. 5,013,556. Albumin nanoparticles are particularly preferred in the compositions of the present invention.

Particularly useful liposomes can be generated by, for example, the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Polynucleotides of the present invention can be conjugated to the liposomes using methods as described in Werle et al., Int. J. Pharm. 370(1-2): 26-32 (2009).

The invention further provides for the use of Cell-Penetrating Peptides (CPPs) to facilitate the delivery of the SPARC antisense molecules disclosed herein. CPPs are peptides that are able to efficiently penetrate cellular lipid bilayers. Because of this feature, they can be used to obtain alterations in gene expression. CPPs have been utilized in in vivo and in vitro experiments as delivery vectors for different bioactive cargoes. In particular. CPPs have been used as vectors for multiple effectors of gene expression such as oligonucleotides for antisense, siRNA (small interfering RNA) and decoy dsDNA (double-stranded DNA) applications, and as transfection agents for plasmid delivery. Any suitable conjugation method may be employed to couple the CPP and the oligonucleotide (Heitz et al., Br J. Pharmacol. 2009 157(2):195-206.) Suitable CPPs include, but are not limited to, Tat, Penetratin, Transportan, VP-22, MPG, Pep-1, MAP, PPTG1, SAP, Oligoarginine, SynB, Pvec, and hCT (9-32) (Heitz et al., Br J. Pharmacol. 2009 157(2):195-206.).

In other embodiments, a composition can be delivered using a natural virus or virus-like particle, a dendrimer, carbon nanoassembly, a polymer carrier, a paramagnetic particle, a ferromagnetic particle, a polymersome, a filomicelle, a micelle or a lipoprotein.

Administration into the airways can provide either systemic or local administration, for example to the trachea and/or the lungs. Such administration can be made via inhalation or via physical application, using aerosols, solutions, and devices such as a bronchoscope. For inhalation, the compositions herein are conveniently delivered from an insufflator, a nebulizer, a pump, a pressurized pack, or other convenient means of delivering an aerosol, non-aerosol spray of a powder, or noon-aerosol spray of a liquid. Pressurized packs can comprise a suitable propellant such a liquefied gas or a compressed gas. Liquefied gases include, for example, fluorinated chlorinated hydrocarbons, hydrochlorofluorocarbons, hydrochlorocarbons, hydrocarbons, and hydrocarbon ethers. Compressed gases include, for example, nitrogen, nitrous oxide, and carbon dioxide. In particular, the use of dichlorodifluoromethane, trichlorofluoromethane, dichlorotetrafluoroethane, carbon dioxide or other suitable gas is contemplated. In the case of a pressurized aerosol, the dosage unit can be determined by providing a valve to deliver a controlled amount. In administering a dry powder composition, the powder mix can include a suitable powder base such as lactose or starch. The powder composition can be presented in unit dosage form such as, for example, capsules, cartridges, or blister packs from which the powder can be administered with the aid of an inhalator or insufflator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, inhaled aerosols, rectal or vaginal suppositories, mouthwashes, rapidly dissolving tablets, or lozenges. For transdermal administration, the active compounds are formulated into ointments, salves, gels, foams, or creams as generally known in the art.

The pharmaceutical compositions can be delivered using drug delivery systems. Such delivery systems include hyaluronic acid solutions or suspensions of collagen fragments. The drugs can be formulated in microcapsules, designed with appropriate polymeric materials for controlled release, such as polylactic acid, ethylhydroxycellulose, polycaprolactone, polycaprolactone diol, polylysine, polyglycolic, polymaleic acid, poly[N-(2-hydroxypropyl)methylacrylamide] and the like. Particular formulations using drug delivery systems can be in the form of liquid suspensions, ointments, complexes to a bandage, collagen shield or the like.

The composition can further comprise any other suitable components, especially for enhancing the stability of the composition and/or its end-use. Accordingly, there is a wide variety of suitable formulations of the composition of the invention.

Sustained release compositions can also be employed in the present compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the pharmaceutical composition and physiological distress.

Compositions provided by the invention can include, e.g., from about 0.5 mL to about 4 mL aqueous or organic liquids with an active agent coupled to a SPARC antisense oligonucleotide, with the concentration of the active agent from about 10 mg/mL to about 100 mg/mL, preferably from about 1 mg/mL to about 10 mg/mL, more preferably from about 0.1 mg/mL to about 1 mg/mL. The active agent can be present at any suitable and therapeutically effective concentration, e.g., Avastin at a concentration of from about 10 mg/mL to about 50 mg/mL.

### IV. Methods

The invention provides methods of treating or preventing proliferative diseases in animals comprising administering a therapeutically effective amount of one or more of the SPARC antisense compositions provided by the invention. In some embodiments, the invention provides a method for treating a disease in a mammal comprising administering an effective amount of a composition comprising a SPARC antisense oligonucleotide. Any suitable composition employing any SPARC antisense oligonucleotide described above can be used in the methods of the present invention.

Methods of treating a proliferative disease in an animal in accordance with the invention include, e.g., methods comprising: (a) isolating RNA or protein from lesional tissue in the animal, (b) isolating RNA or protein from corresponding normal tissue, (c) measuring the level of SPARC RNA or protein said lesional tissue, (d) measuring the level of SPARC RNA or protein in said corresponding normal tissue, (e) comparing the level of SPARC RNA or proein in said lesional tissue with the level of SPARC RNA in said corresponding normal tissue, and (f) administering a therapeutically effective amount of the SPARC antisense composition of the invention to the animal when the comparison in step (e) indicates that there exists a higher level of SPARC RNA or protein in the lesional tissue relative to the level of SPARC RNA in the corresponding normal tissue. The level of SPARC RNA can be determined by any suitable technique, including, e.g, in situ hybridization, blot hybridization, PCR, TMA, invader or microarray. The level of SPARC protein can be determined by any suitable technique, including, e.g, immunohistology, immunoblot, antibody microarray or mass spectroscopy.

Alternatively, methods of treating a proliferative disease in an animal in accordance with the invention include methods which do not determining or comparing the level of SPARC RNA or protein in the lesion.

According to the methods of the present invention, a therapeutically effective amount of the composition can be administered to the mammal to enhance delivery of the active agent to a disease site relative to delivery of the active agent alone, or to enhance clearance resulting in a decrease in blood level of SPARC. In preferred embodiments, the decrease in blood level of SPARC is at least about 10%. In more preferred embodiments, the decrease in blood level of SPARC is at least about 15%, 20%, 25%, 30%, 35%, 40%, 45%, or, most preferably, at least about 50%.

The present methods can be used in any condition characterized by overexpression of SPARC. Exemplary diseases for which the present invention is useful include abnormal conditions of proliferation, tissue remodeling, hyperplasia, exaggerated wound healing in any bodily tissue including soft tissue, connective tissue, bone, solid organs, blood vessel and the like. Examples of diseases treatable or diagnosed using the methods and compositions of the present invention include cancer, diabetic or other retinopathy, inflammation, arthritis, restenosis in blood vessels or artificial blood vessel grafts or intravascular devices and the like.

Suitable proliferative diseases for treatment or prevention in accordance with the invention include, without limitation, cancer, restenosis or other proliferative diseases, fibrosis, osteoporosis or exaggerated wound healing. Specifically, such suitable diseases include, without limitation, wherein: (a) the cancer is selected from the group consisting of circinoma in situ, atypical hyperplasia, carcinoma, sarcoma, carcinosarcoma, lung cancer, pancreatic cancer, skin cancer, melanoma, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer, (b) the restenosis is selected from the group consisting of coronary artery restenosis, cerebral artery restenosis, carotid artery restenosis, renal artery restenosis, femoral artery restenosis, peripheral artery restenosis or combinations thereof, (c) the other proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas, and (d) the fibrotic disease is selected from the group consisting of hepatic fibrosis, pulmonary fibrosis and retroperitoneal fibrosis.

Methods in accordance with the invention include, without limitation, those in which the SPARC antisense composition is administered directly to the diseased tissue in the organism, intravenously, subcutaneously, intramuscularly, nasally, intraperitonealy, vagainally, anally, orally, intraocularly or intrathecally. Methods in accordance with the invention include, e.g., combination therapies wherein the animal is also undergoing one or more cancer therapies selected from the group consisting of surgery, chemotherapy, radiotherapy, thermotherapy, immunotherapy, hormone therapy and laser therapy.

One or more doses of one or more chemotherapeutic agents, such as those described above, can also be administered according to the inventive methods. The type and number of chemotherapeutic agents used in the inventive method will depend on the standard chemotherapeutic regimen for a particular tumor type. In other words, while a particular cancer can be treated routinely with a single chemotherapeutic agent, another can be treated routinely with a combination of chemotherapeutic agents. Methods for combination therapies employing suitable therapeutics, chemotherapeutics, radionuclides, etc. to antibodies or fragments thereof are well described in the art.

In general any combination therapy will include one or more of chemotherapeutics, targeting agents like antibodies; kinase inhibitors; hormonal agents and the like. Combination therapies can also include conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, anti-cancer agents that are well known in the art and can be used as a treatment in combination with the compositions described herein include, but are not limited to As used herein, a first line “chemotherapeutic agent” or first line chemotherapy is a medicament that may be used to treat cancer, and generally has the ability to kill cancerous cells directly. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

As used herein the term “alternative therapeutic regimen” or “alternative therapy” (not a first line chemotherapeutic regimen as described above) may include for example, receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rouse sarcoma virus transforming oncogene (SRC) inhibitors, histonedeacetylase inhibitors, small hypoxia-inducible factor inhibitors, hedgehog inhibitors, and TGF-13 signalling inhibitors. Furthermore, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Methods of use of chemotherapeutic agents and other agents used in alternative therapeutic regimens in combination therapies, including dosing and administration regimens, will also be known to a physician versed in the art.

In order for an antisense oligonucleotide to down-regulate gene expression, it must penetrate into the targeted cells. Uptake occurs through active transport, which in turn depends on temperature, the structure and the concentration of the oligonucleotide, and the cell line. Without desiring to be bound by any theories of the mechanism of action, it is believed that adsorptive endocytosis and fluid phase pinocytosis are the major mechanisms of oligonucleotide internalization, with the relative proportions of internalized material depending on oligonucleotide concentration. At relatively low oligonucleotide concentration, it is likely that internalization occurs via interaction with a membrane-bound receptor. At relatively high oligonucleotide concentration, these receptors are saturated, and the pinocytotic process assumes larger importance.

The use of vectors in antisense drug delivery in accordance with the invention is optional. Clinical trials with antisense oligonucleotides are carried out with naked oligonucleotides.

However to improve cellular uptake and oligonucleotide spatial and temporal activity, a range of techniques and vectors have been developed. Suitable vectors include liposomes, which are vesicular colloid vesicles generally composed of bilayers of phospholipids and cholesterol. Liposomes can be neutral or cationic, depending on the nature of the phospholipids. The oligonucleotide can be easily encapsulated in the liposome interior, which contains an aqueous compartment, or be bound to the liposome surface by electrostatic interactions. These vectors, because of their positive charge, have high affinity for cell membranes, which are negatively charged under physiological conditions. As these vectors use the endosomal pathway to deliver oligonucleotides into cells, certain “helper” molecules have been added into the liposomes to allow the oligonucleotides to escape from the endosomes; these include species such as chloroquine and 1,2-dioleoyl-sn-glycero-3-phosphatidylethanolamine. These “helper” molecules ultimately induce endosomal membrane destabilization, allowing leakage of the oligonucleotide, which then appears to be actively transported in high concentration to the nucleus. Many commercial vectors, such as Lipofectin and compounds known collectively as Eufectins, Cytofectin, Lipofectamine, etc., are commonly used in laboratory research studies. With some of these delivery vehicles, and under defined conditions, oligonucleotide concentrations of <50 nm may be successfully used. The use of other cationic polymers, including, e.g., poly-L-lysine, PAMAM dendrimers, polyalkylcyanoacrylate nanoparticles, CPPs, and polyethyleneimine, are also suitable for use in accordance with the invention.

All of these cationic delivery systems internalize oligonucleotides via an endocytosic mechanism. To avoid the resulting compartmentalization problems, consideration has been given to modulating plasma membrane permeability. By using basic peptides, one can increase oligonucleotide passage through the plasma membrane by a receptor- and transporter-independent mechanism. As these peptides have membrane translocation properties, covalent coupling with an oligonucleotide can increase the latter's penetration into the cell, delivering them directly into the cytoplasm and hence ultimately the nucleus.

An additional suitable approach to oligonucleotide internalization is to generate transient permeabilization of the plasma membrane and allow naked oligonucleotides to penetrate into the cells by diffusion. This approach involves the formation of transitory pores in the membrane, induced either chemically by streptolysin O permeabilization, mechanically by microinjection or scrape loading, or produced by electroporation.

Compositions in accordance with the invention can be formulated in combination with another agent, e.g., another therapeutic agent or an agent that stabilizes an oligonucleotide agent, e.g., a protein which complexes with the oligonucleotide agent. Still other agents include, without limitation, chelators, salts, and RNAse inhibitors (e.g., RNAsin).

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic.

The oligonucleotide agents featured in the invention can include a delivery vehicle, such as liposomes, for administration to a subject, carriers and diluents and their salts, and/or can be present in pharmaceutically acceptable formulations. Methods for the delivery of nucleic acid molecules are well known in the art.

Pharmaceutical compositions featured in the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized. Preferred physiologically acceptable carrier media are water, buffered water, normal saline, 0.4% saline, 0.3% glycine, hyaluronic acid and the like.

The present invention also features compositions prepared for storage or administration that include a pharmaceutically effective amount of the desired oligonucleotides in a pharmaceutically acceptable carrier or diluent. Acceptable carriers or diluents for therapeutic use are well known in the pharmaceutical art.

Sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

Pharmaceutical compositions of the invention can be administered in a single dose or in multiple doses. Where the administration of such a composition is by infusion, the infusion can be a single sustained dose or can be delivered by multiple infusions. Injection of the agent can be directly into the tissue at or near the site of aberrant target gene expression. Multiple injections of the agent can be made into the tissue at or near the site.

Dosage levels on the order of about 1 ug/kg to 100 mg/kg of body weight per administration are useful in the treatment of a disease. In regard to dosage, an compositions of the present invention can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antisense composition per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antisense composition per kg of bodyweight. The unit dose, for example, can be administered by injection (e.g., intravenous or intramuscular, intrathecally, or directly into an organ), inhalation, or a topical application.

One skilled in the art can also readily determine an appropriate dosage regimen for administering the antisense composition of the invention to a given subject. In some embodiments, the compositions are administered once or twice daily to a subject for a period of from about three to about twenty-eight days, more preferably from about seven to about ten days. In further embodiments, the unit dose is administered less frequently than once a day, e.g., less than every 2, 4, 8 or 30 days. In other embodiments, the unit dose is not administered with a frequency (e.g., not a regular frequency). In another embodiment, the unit dose is not administered with a frequency (e.g., not a regular frequency). In other embodiments, the SPARC antisense composition can be administered to the subject once, as a single injection or deposition at or near the site on unwanted target nucleic acid expression. Because oligonucleotide agent-mediated up-regulation can persist for several days after administering the antisense composition, in many instances, it is possible to administer the composition with a frequency of less than once per day, or, for some instances, only once for the entire therapeutic regimen.

Where a dosage regimen comprises multiple administrations, it is understood that the effective amount of SPARC antisense composition administered to the subject can include the total amount of antisense composition administered over the entire dosage regimen. One skilled in the art will appreciate that the exact individual dosages may be adjusted somewhat depending on a variety of factors, including the specific SPARC antisense composition being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. Following successful treatment, it may be desirable to have the patient undergo maintenance therapy to prevent the recurrence of the disease state. The concentration of the antisense composition is an amount sufficient to be effective in treating or preventing a disorder or to regulate a physiological condition in humans. The concentration or amount of antisense composition administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. It will also be appreciated that the effective dosage of the antisense composition used for treatment may increase or decrease over the course of a particular treatment. Changes in dosage may result and become apparent from the results of diagnostic assays. For example, the subject can be monitored after administering an antisense composition. Based on information from the monitoring, an additional amount of the antisense composition can be administered. Persons of ordinary skill can easily determine optimum dosages, dosing methodologies and repetition rates.

The animal can be any patient or subject in nead of treatment or diagnosis. In preferred embodiments, the animal is a mammal. In particularly preferred embodiments, the animal is a human. In other embodiments, the animal can be a mouse, rat, rabbit, cat, dog, pig, sheep, horse, cow, or a non-human primate.

The following examples further illustrate the invention but, of course, should not be construed as in any way limiting its scope.

### Example 1

This example demonstrates the construction of SPARC-GFP reporter line.

PCR was used to amplify the BIO1 SPARC open reading frame (ORF), with simultaneous introduction of N terminal Kozak sequence, C-terminal 6×His-tag. The product was cloned by TOPO-TA cloning into C-terminal GFP fusion TOPO-TA expression vector. The resulting plasmid, pXL39-Biol-GFP, was transfected into 293 cells by lipofectamine. Forty eight hours later, the cells were selected by 1 mg/ml G418 and the single clones were picked and screened for best GFP signal.

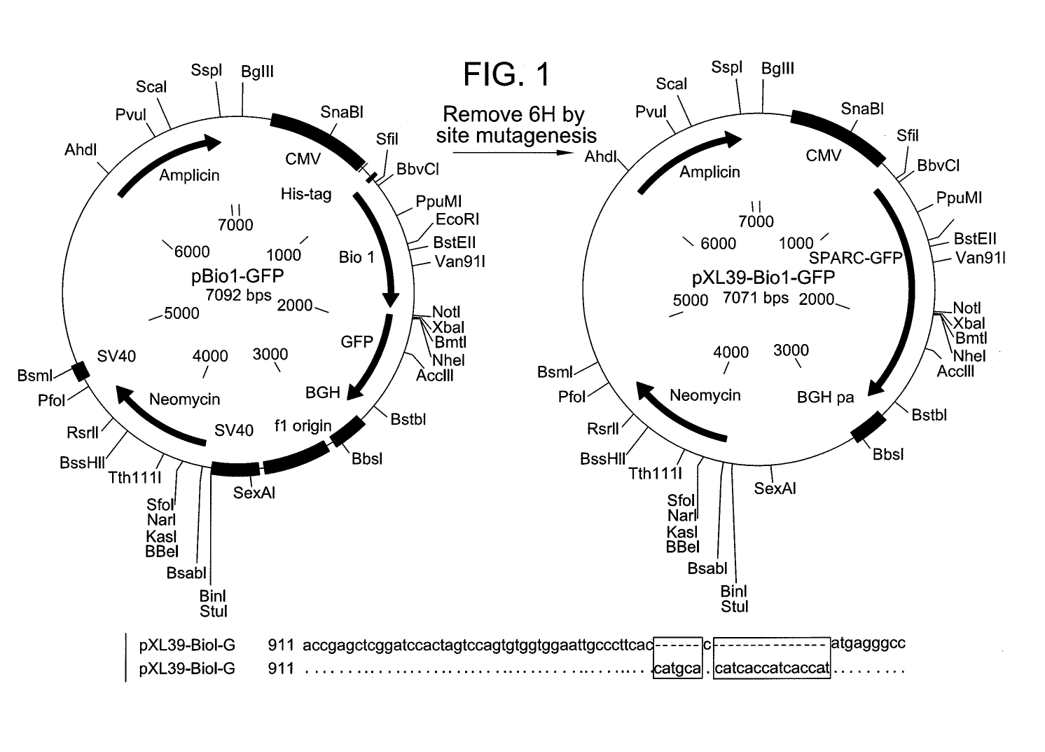

The map of the resulting pXL39-Biol-GFP is shown in FIG. 1.

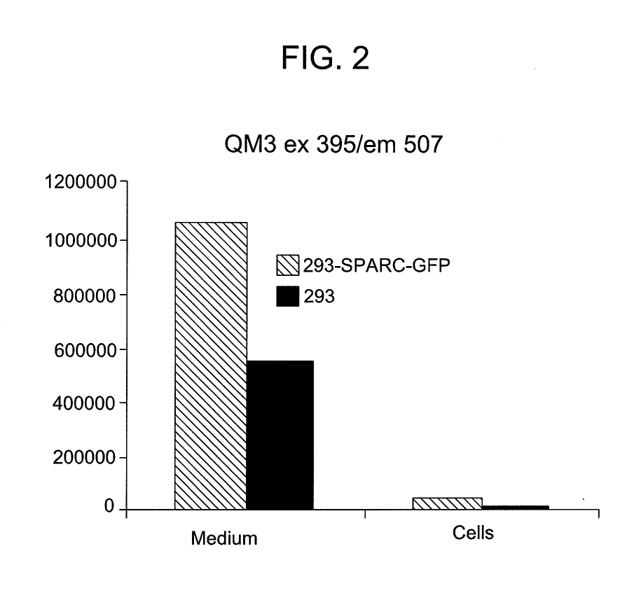

Next, 293 cells stably transfected with pXL39-Biol-GFP were tested for GFP signal. Cells were cultured in DMEM/FBS/G418 from T175 for 3 days and GFP measurement was performed on the conditioned media and the cells. The 10⁶ cells were washed 2× with HBSS prior to assay. Measurement was performed using the QM3 with excitation 395 nm, emission peak 510 nm. As shown in FIG. 2, significantly greater GFP activity was observed for SPARC-GFP reporter line than control, untransfected cells.

Clones were generated from the transfected cells and applied at a starting concentration of 100 nM, with 0.5 μL/well Dharmafect™ (Dharmacon, Inc., Lafayette, Colo.) or Lipofectamine™ (Invitrogen, Carlsbad, Calif.) transfection reagents, to conditioned media (F12-K medium, to which 0.375% FBS, 0.25×NEAA, and 0.25 mg/mL G418 were added during transfection complex formation) optimized to minimize background fluorescence (data not shown). Clones were evaluated for GFP signal and stability at three days and one month (data not shown), and clone AL2-11 was selected. Cells from the selected AL2-11 cell line were seeded at 50K/well, and incubated for 24 and 48 hours.

GFP signals and stability were detected by victor 3 plate reader and QM3, with exemplary results shown at FIG. 2.

### Example 2

This example demonstrates the measurement of the decrease in BIO1 SPARC-GFP signal (“knock-down activity”) in response to siRNA and antisense oligonucleotides.

Three anti-SPARC siRNA sequences, si13347, si13346, si13345 (SEQ ID NOs: 201-203, respectively), were commercially obtained from Ambion (Austin, Tex.). Additional antisense oligonucleotides were similarly prepared (SEQ ID NOs: 14-70) using walk-through analysis of the SPARC open reading frame. Corresponding sense oligonucleotides (SEQ ID NOS: 15-127) were prepared for use as negative controls. A second library of antisense oligonucleotides (SEQ ID NOs: 128-200) including LNAs was also prepared.

SEQ ID NOs: 15-203 are provided in Tables 2-5 below. (* indicate PS, + indicate LNA):

TABLE 2

| SEQ | | |
|---|---|---|
| ID | | Sequence |
| NO: | Sequence | designation |
| 201 | ACTCCATAGACACCCTCGA | si13347 |
| 202 | GATGTAGCCCGGAACGTTTAT | si13346 |
| 203 | TCCCTACCTCCTGTTGTTGGAA | si13345 |

TABLE 3

| SEQ | | |
|---|---|---|
| ID | | Sequence |
| NO: | Sequence | designation |
| 14 | +T + T*A*G* + A + T*C*A* + C + A*A*G* + A + T*C*C | anti-SP-1 |
| 15 | +T + T*G*T* + C + G*A*T* + A + T*C*C* + T + T*C*T | anti-SP-2 |
| 16 | +G + C*T*T* + G + A*T*G* + C + C*G*A* + A + G*C*A | anti-SP-3 |
| 17 | +G + C*C*G* + G + C*C*C* + A + C*T*C* + A + T*C*C | anti-SP-4 |
| 18 | +A + G*G*G* + C + G*A*T* + G + T*A*C* + T + T*G*T | anti-SP-5 |
| 19 | +C + A*T*T* + G + T*C*C* + A + G*G*T* + C + A*C*A | anti-SP-6 |
| 20 | +G + G*T*C* + T + C*G*A* + A + A*A*A* + G + C*G*G | anti-SP-7 |
| 21 | +G + T*G*G* + T + G*C*A* + A + T*G*C* + T + C*C*A | anti-SP-8 |
| 22 | +T + G*G*G* + G + A*T*G* + A + G*G*G* + G + A*G*C | anti-SP-9 |
| 23 | +A + C*G*C* + A + G*T*G* + G + A*G*C* + C + A*G*C | anti-SP-10 |
| 24 | +T + C*G*G* + T + G*T*G* + G + G*A*G* + A + G*G*T | anti-SP-11 |
| 25 | +A + C*C*C* + G + T*C*A* + A + T*G*G* + G + G*T*G | anti-SP-12 |
| 26 | +C + T*G*G* + T + C*C*A* + G + C*T*G* + G + C*C*G* | anti-SP-13 |
| 27 | +A + A*C*T* + G + C*C*A* + G + T*G*T* + A + C*A*G | anti-SP-14 |
| 28 | +G + G*A*A* + G + A*T*G* + T + A*C*A* + T + G*T*T | anti-SP-15 |
| 29 | +A + T*A*G* + T + T*C*T* + T + C*T*C* + G + A*A*G | anti-SP-16 |
| 30 | +T + C*C*C* + G + G*G*C* + C + A*G*C* + A + G*C*T | anti-SP-17 |
| 31 | +C + C*A*C* + G + G*G*G* + T + G*G*T* + C + T*C*C | anti-SP-18 |
| 32 | +T + G*C*C* + T + C*C*A* + G + G*C*G* + C + T*T*C | anti-SP-19 |
| 33 | +T + C*A*T* + T + C*T*C* + A + T*G*G* + A + T*C*T | anti-SP-20 |
| 34 | +T + C*T*T* + C + A*C*C* + C + G*C*A* + G + C*T*T | anti-SP-21 |
| 35 | +C + T*G*C* + T + T*C*T* + C + A*G*T* + C + A*G*A | anti-SP-22 |
| 36 | +A + G*G*T* + T + G*T*T* + G + T*C*C* + T + C*A*T | anti-SP-23 |
| 37 | +C + C*C*T* + C + T*C*A* + T + A*C*A* + G + G*G*T | anti-SP-24 |
| 38 | +G + A*C*C* + A + G*G*A* + C + G*T*T* + C + T*T*G | anti-SP-25 |
| 39 | +A + G*C*C* + A + G*T*C* + C + C*G*C* + A + T*G*C | anti-SP-26 |
| 40 | +G + C*A*G* + G + G*G*G* + A + A*T*T* + C + G*G*T | anti-SP-27 |
| 41 | +C + A*G*C* + T + C*A*G* + A + G*T*C* + C + A*G*G | anti-SP-28 |
| 42 | +C + A*A*G* + G + G*G*G* + G + A*T*G* + T + A*T*T | anti-SP-29 |
| 43 | +T + G*C*A* + A + G*G*C* + C + C*G*A* + T + G*T*A | anti-SP-30 |
| 44 | +G + T*C*C* + A + G*G*T* + G + G*A*G* + C + T*T*G | anti-SP-31 |
| 45 | +T + G*G*C* + C + C*T*T* + C + T*T*G* + G + T*G*C | anti-SP-32 |
| 46 | +C + C*T*C* + C + A*G*G* + G + T*G*C* + A + C*T*T | anti-SP-33 |
| 47 | +T + G*T*G* + G + C*A*A* + A + G*A*A* + G + T*G*G | anti-SP-34 |
| 48 | +C + A*G*G* + A + A*G*A* + G + T*C*G* + A + A*G*G | anti-SP-35 |
| 49 | +T + C*T*T* + G + T*T*G* + T + C*A*T* + T + G*C*T | anti-SP-36 |
| 50 | +G + C*A*C* + A + C*C*T* + T + C*T*C* + A + A*A*C | anti-SP-37 |
| 51 | +T + C*G*C* + C + A*A*T* + G + G*G*G* + G + C*T*G | anti-SP-38 |
| 52 | +G + G*C*A* + G + C*T*G* + G + T*G*G* + G + G*T*C | anti-SP-39 |
| 53 | +C + T*G*G* + C + A*C*A* + C + G*C*A* + C + A*T*G | anti-SP-40 |
| 54 | +G + G*G*G* + T + G*T*T* + G + T*T*C* + T + C*A*T | anti-SP-41 |
| 55 | +C + C*A*G* + C + T*C*G* + C + A*C*A* + C + C*T*T | anti-SP-42 |
| 56 | +G + C*C*G* + T + G*T*T* + T + G*C*A* + G + T*G*G | anti-SP-43 |
| 57 | +T + G*G*T* + T + C*T*G* + G + C*A*G* + G + G*A*T | anti-SP-44 |
| 58 | +T + T*T*C* + C + G*C*C* + A + C*C*A* + C + C*T*C | anti-SP-45 |
| 59 | +C + T*C*T* + T + C*G*G* + T + T*T*C* + C + T*C*T | anti-SP-46 |
| 60 | +G + C*A*C* + C + A*T*C* + A + T*C*A* + A + A*T*T | anti-SP-47 |
| 61 | +C + T*C*C* + T + A*C*T* + T + C*C*A* + C + C*T*G | anti-SP-48 |
| 62 | +G + A*C*A* + G + G*A*T* + T + A*G*C* + T + C*C*C | anti-SP-49 |
| 63 | +A + C*A*G* + A + T*A*C* + C + T*C*A* + G + T*C*A | anti-SP-50 |
| 64 | +C + C*T*C* + T + G*C*C* + A + C*A*G* + T + T*T*C | anti-SP-51 |
| 65 | +T + T*C*C* + A + C*C*A* + C + C*T*C* + T + G*T*C | anti-SP-52 |
| 66 | +T + C*A*T* + C + A*G*G* + C + A*G*G* + G + C*T*T | anti-SP-53 |
| 67 | +C + T*T*G* + C + T*G*A* + G + G*G*G* + C + T*G*C | anti-SP-54 |
| 68 | +C + A*A*G* + G + C*C*C* + T + C*C*C* + G + G*C*C | anti-SP-55 |
| 69 | +A + G*G*C* + A + A*A*G* + G + A*G*A* + A + A*G*A | anti-SP-56 |
| 70 | +A + G*A*T* + C + C*A*G* + G + C*C*C* + T + C*A*T | anti-SP-57 |

TABLE 4

| SEQ | | |
|---|---|---|
| ID | | Sequence |
| NO: | Sequence | designation |
| 71 | +A + T*G*A* + G + G*G*C* + C + T*G*G* + A + T*C*T | AZ-SP-1 |
| 72 | +T + C*T*T* + T + C*T*C* + C + T*T*T* + G + C*C*T | AZ-SP-2 |
| 73 | +G + G*C*C* + G + G*G*A* + G + G*G*C* + C + T*T*G | AZ-SP-3 |
| 74 | +G + C*A*G* + C + C*C*C* + T + C*A*G* + C + A*A*G | AZ-SP-4 |
| 75 | +A + A*G*C* + C + C*T*G* + C + C*T*G* + A + T*G*A | AZ-SP-5 |
| 76 | +G + A*C*A* + G + A*G*G* + T + G*G*T* + G + G*A*A | AZ-SP-6 |
| 77 | +G + A*A*A* + C + T*G*T* + G + G*C*A* + G + A*G*G | AZ-SP-7 |
| 78 | +T + G*A*C* + T + G*A*G* + G + T*A*T* + C + T*G*T | AZ-SP-8 |
| 79 | +G + G*G*A* + G + C*T*A* + A + T*C*C* + T + G*T*C | AZ-SP-9 |
| 80 | +C + A*G*G* + T + G*G*A* + A + G*T*A* + G + G*A*G | AZ-SP-10 |
| 81 | +A + A*T*T* + T + G*A*T* + G + A*T*G* + G + T*G*C | AZ-SP-11 |
| 82 | +A + G*A*G* + G + A*A*A* + C + C*G*A* + A + G*A*G | AZ-SP-12 |
| 83 | +G + A*G*G* + T + G*G*T* + G + G*C*G* + G + A*A*A | AZ-SP-13 |
| 84 | +A + T*C*C* + C + T*G*C* + C + A*G*A* + A + C*C*A | AZ-SP-14 |
| 85 | +C + C*A*C* + T + G*C*A* + A + A*C*A* + C + G*G*C | AZ-SP-15 |
| 86 | +A + A*G*G* + T + G*T*G* + C + G*A*G* + C + T*G*G | AZ-SP-16 |
| 87 | +A + T*G*A* + G + A*A*C* + A + A*C*A* + C + C*C*C | AZ-SP-17 |
| 88 | +C + A*T*G* + T + G*C*G* + T + G*T*G* + C + C*A*G | AZ-SP-18 |
| 89 | +G + A*C*C* + C + C*A*C* + C + A*G*C* + T + G*C*C | AZ-SP-19 |
| 90 | +C + A*G*C* + C + C*C*C* + A + T*T*G* + G + C*G*A | AZ-SP-20 |
| 91 | +G + T*T*T* + G + A*G*A* + A + G*G*T* + G + T*G*C | AZ-SP-21 |
| 92 | +A + G*C*A* + A + T*G*A* + C + A*A*C* + A + A*G*A | AZ-SP-22 |
| 93 | +C + C*T*T* + C + G*A*C* + T + C*T*T* + C + C*T*G | AZ-SP-23 |
| 94 | +C + C*A*C* + T + T*C*T* + T + T*G*C* + C + A*C*A | AZ-SP-24 |
| 95 | +A + A*G*T* + G + C*A*C* + C + C*T*G* + G + A*G*G | AZ-SP-25 |
| 96 | +G + C*A*C* + C + A*A*G* + A + A*G*G* + G + C*C*A | AZ-SP-26 |
| 97 | +C + A*A*G* + C + T*C*C* + A + C*C*T* + G + G*A*C | AZ-SP-27 |
| 98 | +T + A*C*A* + T + C*G*G* + G + C*C*T* + T + G*C*A | AZ-SP-28 |
| 99 | +A + A*T*A* + C + A*T*C* + C + C*C*C* + C + T*T*G | AZ-SP-29 |
| 100 | +C + C*T*G* + G + A*C*T* + C + T*G*A* + G + C*T*G | AZ-SP-30 |
| 101 | +A + C*C*G* + A + A*T*T* + C + C*C*C* + C*T*G*C | AZ-SP-31 |
| 102 | +G + C*A*T* + G + C*G*G* + G + A*C*T* + G + G*C*T | AZ-SP-32 |
| 103 | +C + A*A*G* + A + A*C*G* + T + C*C*T* + G + G*T*C | AZ-SP-33 |
| 104 | +A + C*C*C* + T + G*T*A* + T + G*A*G* + A + G*G*G | AZ-SP-34 |
| 105 | +A + T*G*A* + G + G*A*C* + A + A*C*A* + A + C*C*T | AZ-SP-35 |
| 106 | +T + C*T*G* + A + C*T*G* + A + G*A*A* + G + C*A*G | AZ-SP-36 |
| 107 | +A + A*G*C* + T + G*C*G* + G + G*T*G* + A + A*G*A | AZ-SP-37 |
| 108 | +A + G*A*T* + C + C*A*T* + G + A*G*A* + A + T*G*A | AZ-SP-38 |
| 109 | +G + A*A*G* + C + G*C*C* + T + G*G*A* + G + G*C*A | AZ-SP-39 |
| 110 | +G + G*A*G* + A + C*C*A* + C + C*C*C* + G + T*G*G | AZ-SP-40 |
| 111 | +A + G*C*T* + G + C*T*G* + G + C*C*C* + G + G*G*A | AZ-SP-41 |
| 112 | +C + T*T*C* + G + A*G*A* + A + G*A*A* + C + T*A*T | AZ-SP-42 |
| 113 | +A + A*C*A* + T + G*T*A* + C + A*T*C* + T + T*C*C | AZ-SP-43 |
| 114 | +C + T*G*T* + A + C*A*C* + T + G*G*C* + A + G*T*T | AZ-SP-44 |
| 115 | +C + G*G*C* + C + A*G*C* + T + G*G*A* + C + C*A*G | AZ-SP-45 |
| 116 | +C + A*C*C* + C + C*A*T* + T + G*A*C* + G + G*G*T | AZ-SP-46 |
| 117 | +A + C*C*T* + C + T*C*C* + C + A*C*A* + C + C*G*A | AZ-SP-47 |
| 118 | +6 + C*T*G* + G + C*T*C* + C + A*C*T* + G + C*G*T | AZ-SP-48 |
| 119 | +G + C*T*C* + C + C*C*T* + C + A*T*C* + C + C*C*A | AZ-SP-49 |
| 120 | +T + G*G*A* + G + C*A*T* + T + G*C*A* + C + C*A*C | AZ-SP-50 |
| 121 | +C + C*G*C* + T + T*T*T* + T + C*G*A* + G + A*C*C | AZ-SP-51 |
| 122 | +T + G*T*G* + A + C*C*T* + G + G*A*C* + A*A*T*G | AZ-SP-52 |
| 123 | +A + C*A*A* + G + T*A*C* + A + T*C*G* + C + C*C*T | AZ-SP-53 |
| 124 | +G + G*A*T* + G + A*G*T* + G + G*G*C* + C + G*G*C | AZ-SP-54 |
| 125 | +T + G*C*T* + T + C*G*G* + C + A*T*C* + A + A*G*C | AZ-SP-55 |
| 126 | +A + G*A*A* + G + G*A*T* + A + T*C*G* + A + C*A*A | AZ-SP-56 |
| 127 | +G + G*A*T* + C + T*T*G* + T + G*A*T* + C + T*A*A | AZ-SP-57 |

TABLE 5

| SEQ | | |
|---|---|---|
| ID | | Sequence |
| NO: | Sequence | designation |
| 128 | +G* + G*G*T* + T* + T*A*G* + A* + G*A*C* + A* + G*G*C*A* + A* + C | AS-SPARC-20 |
| 129 | +G* + G*A*C* + C* + G*C*G* + G* + G*A*A* + T* + G*T*G*G* + A* + G | AS-SPARC-21 |
| 130 | +G* + C*T*C* + T* + C*C*G* + G* + G*C*A* + G* + T*C*T*G* + A* + A | AS-SPARC-22 |
| 131 | +C* + A*G*G* + C* + G*G*C* + A* + G*G*C* + A* + G*A*G*C* + G* + C | AS-SPARC-23 |
| 132 | +A* + C*C*C* + T* + C*A*G* + T* + G*G*C* + A* + G*G*C*A* + G* + G | AS-SPARC-24 |
| 133 | +G* + G* + C* + C* + C*T*C*A*T*G*G*T*G*C* + T* + G* + G* + G* + A | AS-SPARC-25 |
| 134 | +A* + A* + A* + G* + G*A*G*A*A*A*G*A*A*G* + A* + T* + C* + C* + A | AS-SPARC-26 |
| 135 | +A* + A* + G* + G* + C*C*C*T*C*C*C*G*G*C* + C* + A* + G* + G* + C | AS-SPARC-27 |
| 136 | +T* + T* + C* + T* + T*G*C*T*G*A*G*G*G*G* + C* + T* + G* + C* + C | AS-SPARC-28 |
| 137 | +C* + T* + G* + T* + C*T*C*A*T*C*A*G*G*C* + A* + G* + G* + G* + C | AS-SPARC-29 |
| 138 | +A* + C* + A* + G* + T*T*T*C*T*T*C*C*A*C* + C* + A* + C* + C* + T | AS-SPARC-30 |
| 139 | +T* + A* + C* + C* + T*C*A*G*T*C*A*C*C*T* + C* + T* + G* + C* + C | AS-SPARC-31 |
| 140 | +T* + C* + T* + C* + C*T*A*C*T*T*C*C*A*C* + C* + T* + G* + G* + A | AS-SPARC-33 |
| 141 | +C* + T* + C* + T* + G*C*A*C*C*A*T*C*A*T* + C* + A* + A* + A* + T | AS-SPARC-34 |
| 142 | +C* + C* + A* + C* + C*T*C*C*T*C*T*T*C*G* + G* + T* + T* + T* + C | AS-SPARC-35 |
| 143 | +T* + G* + G* + C* + A*G*G*G*A*T*T*T*T*C* + C* + G* + C* + C* + A | AS-SPARC-36 |
| 144 | +G* + T* + G* + T* + T*T*G*C*A*G*T*G*G*T* + G* + G* + T* + T* + C | AS-SPARC-37 |
| 145 | +C* + C* + A* + G* + C*T*C*G*C*A*C*A*C*C* + T* + T* + G* + C* + C | AS-SPARC-38 |
| 146 | +A* + T* + G* + G* + G*G*G*T*G*T*T*G*T*T* + C* + T* + C* + A* + T | AS-SPARC-39 |
| 147 | +G* + G* + G* + G* + T*C*C*T*G*G*C*A*C*A* + C* + G* + C* + A* + C | AS-SPARC-40 |
| 148 | +T* + G* + G* + G* + G*G*C*T*G*G*G*C*A*G* + C* + T* + G* + G* + T | AS-SPARC-41 |
| 149 | +A* + C* + C* + T* + T*C*T*C*A*A*A*C*T*C* + G* + C* + C* + A* + A | AS-SPARC-42 |
| 150 | +C* + T* + T* + G* + T*T*G*T*C*A*T*T*G*C* + T* + G* + C* + A* + C | AS-SPARC-43 |
| 151 | +G* + G* + C* + A* + G*G*A*A*G*A*G*T*C*G* + A* + A* + G* + G* + T | AS-SPARC-44 |
| 152 | +C* + A* + C* + T* + T*T*G*T*G*G*C*A*A*A* + G* + A* + A* + G* + T | AS-SPARC-45 |
| 153 | +C* + T* + T* + G* + G*T*G*C*C*C*T*C*C*A* + G* + G* + G* + T* + G | AS-SPARC-46 |
| 154 | +G* + G* + T* + G* + G*A*G*C*T*T*G*T*G*G* + C* + C* + C* + T* + T | AS-SPARC-47 |
| 155 | +C* + A* + A* + G* + G*C*C*C*G*A*T*G*T*A* + G* + T* + C* + C* + A | AS-SPARC-48 |
| 156 | +G* + C* + A* + A* + G*G*G*G*G*G*A*T*G*T* + A* + T* + T* + T* + G | AS-SPARC-49 |
| 157 | +C* + G* + G* + T* + C*A*G*C*T*C*A*G*A*G* + T* + C* + C* + A* + G | AS-SPARC-50 |
| 158 | +C* + G* + C* + A* + T*G*C*G*C*A*G*G*G*G* + G* + A* + A* + T* + T | AS-SPARC-51 |
| 159 | +G* + A* + C* + G* + T*T*C*T*T*G*A*G*C*C* + A* + G* + T* + C* + C | AS-SPARC-52 |
| 160 | +T* + C* + T* + C* + A*T*A*C*A*G*G*G*T*G* + A* + C* + C* + A* + G | AS-SPARC-53 |
| 161 | +A* + G* + G* + T* + T*G*T*T*G*T*C*C*T*C* + A* + T* + C* + C* + C | AS-SPARC-54 |
| 162 | +C* + T* + T* + C* + T*G*C*T*T*C*T*C*A*G* + T* + C* + A* + G* + A | AS-SPARC-55 |
| 163 | +G* + G* + A* + T* + C*T*T*C*T*T*C*A*C*C* + C* + G* + C* + A* + G | AS-SPARC-56 |
| 164 | +A* + G* + G* + C* + G*C*T*T*C*T*C*A*T*T* + C* + T* + C* + A* + T | AS-SPARC-57 |
| 165 | +G* + G* + G* + G* + T*G*G*T*C*T*C*C*T*G* + C* + C* + T* + C* + C | AS-SPARC-58 |
| 166 | +C* + C* + C* + G* + G*G*C*C*A*G*C*A*G*C* + T* + C* + C* + A* + C | AS-SPARC-59 |
| 167 | +T* + T* + A* + T* + A*G*T*T*C*T*T*C*T*C* + G* + A* + A* + G* + T | AS-SPARC-60 |
| 168 | +T* + A* + C* + A* + G*G*G*A*A*G*A*T*G*T* + A* + C* + A* + T* + G | AS-SPARC-61 |
| 169 | +G* + C* + T* + G* + G*C*C*G*A*A*C*T*G*C* + C* + A* + G* + T* + G | AS-SPARC-62 |
| 170 | +T* + C* + A* + A* + T*G*G*G*G*T*G*C*T*G* + G* + T* + C* + C* + A | AS-SPARC-63 |
| 171 | +G* + G* + T* + G* + T*G*G*G*A*G*A*G*G*T* + A* + C* + C* + C* + G | AS-SPARC-64 |
| 172 | +C* + A* + C* + G* + C*A*G*T*G*G*A*G*C*C* + A* + G* + C* + T* + C | AS-SPARC-65 |
| 173 | +T* + C* + C* + A* + T*G*G*G*G*A*T*G*A*G* + G* + G* + G* + A* + G | AS-SPARC-66 |
| 174 | +A* + A* + A* + G* + C*G*G*G*T*G*G*T*G*C* + A* + A* + T* + G* + C | AS-SPARC-67 |
| 175 | +C* + C* + A* + G* + G*T*C*A*C*A*G*G*T*C* + T* + C* + G* + A* + A | AS-SPARC-68 |
| 176 | +G* + C* + G* + A* + T*G*T*A*C*T*T*G*T*C* + A* + T* + T* + G* + T | AS-SPARC-69 |
| 177 | +G* + C* + C* + G* + G*C*C*C*A*C*T*C*A*T* + C* + C* + A* + G* + G | AS-SPARC-70 |
| 178 | +T* + C* + T* + G* + C*T*T*G*A*T*G*C*C*G* + A* + A* + G* + C* + A | AS-SPARC-71 |
| 179 | +A* + G* + A* + T* + C*C*T*T*G*T*C*G*A*T* + A* + T* + C* + C* + T | AS-SPARC-72 |
| 180 | +G* + T* + T* + G* + T*T*G*T*C*C*T*C*A*T*C*C* + C* + T* + C* + T* + | AS-SPARC-73 |
| | C | |
| 181 | +G* + T* + T* + C* + T*T*G*A*G*C*C*A*G*T*C* + C* + C* + G* + C* + A | AS-SPARC-74 |
| 182 | +T* + C* + T* + T* + C*C*A*C*C*A*C*C*T*C*T* + G* + T* + C* + T* + C | AS-SPARC-75 |
| 183 | +T* + C* + A* + C* + C*T*C*T*G*C*C*A*C*A* + G* + T* + T* + T* + C | AS-SPARC-1 |
| 184 | +T* + A* + C* + T* + T*C*C*A*C*C*T*G*G*A* + C* + A* + G* + G* + A | AS-SPARC-2 |
| 185 | +C* + A* + T* + C* + A*T*C*A*A*A*T*T*C*T* + C* + C* + T* + A* + C | AS-SPARC-3 |
| 186 | +C* + C* + T* + C* + T*G*C*A*C*C*A*T*C*A* + T* + C* + A* + A* + A | AS-SPARC-4 |
| 187 | +G* + T* + T* + G* + T*C*A*T*T*G*C*T*G*C* + A* + C* + A* + C* + C | AS-SPARC-5 |
| 188 | +G* + T* + C* + G* + A*A*G*G*T*C*T*T*G*T* + T* + G* + T* + C* + A | AS-SPARC-6 |
| 189 | +G* + G* + A* + A* + G*A*G*T*C*G*A*A*G*G* + T* + C* + T* + T* + G | AS-SPARC-7 |
| 190 | +C* + T* + T* + C* + T*C*A*G*T*C*A*G*A*A* + G* + G* + T* + T* + G | AS-SPARC-8 |
| 191 | +C* + T* + G* + C* + T*T*C*T*C*A*G*T*C*A* + G* + A* + A* + G* + G | AS-SPARC-9 |
| 192 | +C* + T* + G* + C* + T*T*C*T*C*A*G*T*C*A* + G* + A* + A* + G* + G | AS-SPARC-10 |
| 193 | +C* + T* + T* + C* + T*C*A*T*T*C*T*C*A*T* + G* + G* + A* + T* + C | AS-SPARC-11 |
| 194 | +T* + A* + C* + A* + G*G*G*A*A*G*A*T*G*T* + A* + C* + A* + T* + G | AS-SPARC-14 |
| 195 | +C* + A* + G* + G* + G*C*G*A*T*G*T*A*C*T* + T* + G* + T* + C* + A | AS-SPARC-15 |
| 196 | +C* + T* + T* + G* + T*C*G*A*T*A*T*C*C*T* + T* + C* + T* + G* + C | AS-SPARC-16 |
| 197 | +T* + T* + A* + G* + C*T*C*C*C*A*C8A*G*A* + T* + A* + C* + C* + T | AS-SPARC-17 |
| 198 | +A* + A* + G* + G* + T*T*G*T*T*G*T*C*C*T* + C* + A* + T* + C* + C | AS-SPARC-18 |
| 199 | +T* + A* + T* + T* + T*G*C*A*A*G*G*C*C*C* + G* + A* + T* + G* + T | AS-SPARC-19 |
| 200 | +T* + C* + A* + T* + C*A*G*G*C*A*G* + G* + G* + C* + T* + T | AS-SP-53-1 |

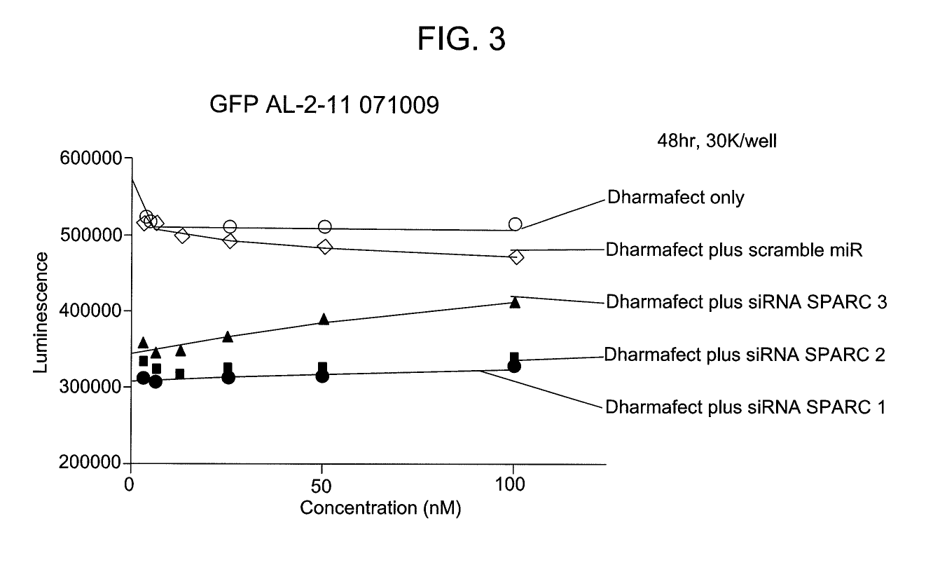

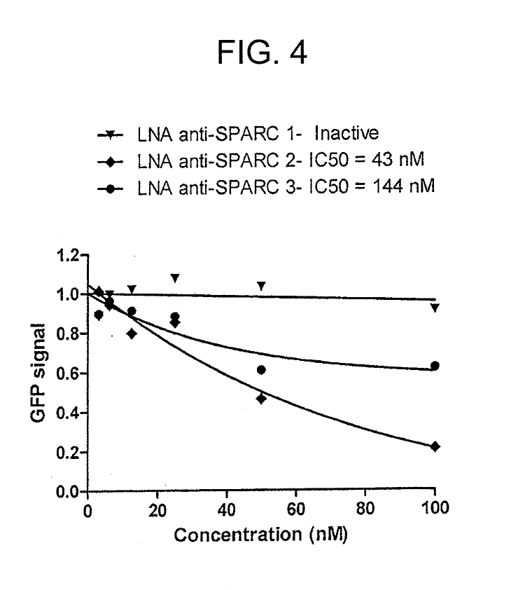

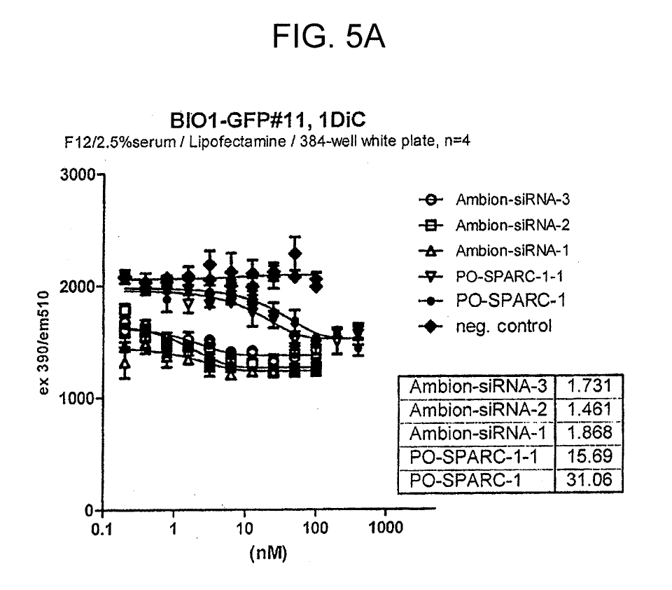

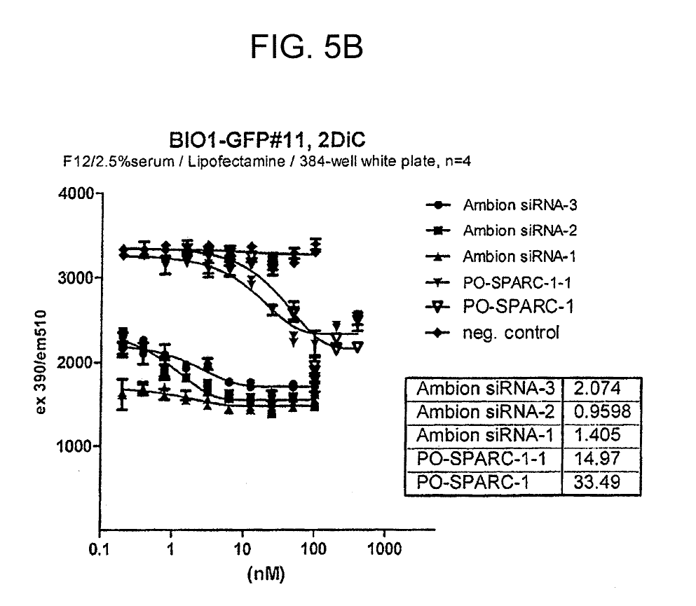

SPARC-GFP reporter cells were transfected with increasing concentrations of the siRNAs and antisense oligonucleotides, from 0.1 nM to 1000 nM, and the GFP signal luminescence was assayed accordingly. As shown in FIG. 3, the siRNAs from Ambion (si13347, si13346, si13345 (SEQ ID NOs: 201-203)) had a high level of knock-down activity against BIO1. As shown in FIG. 4, LNA anti-SPARC-2 (SEQ ID NO:3) and LNA anti-SPARC-3(SEQ ID NO:4) also showed knock-down activity against BIO1 although LNA anti-SPARC-1 (SEQ ID NO:2) was inactive. Antisense oligonucleotides PO-SPARC-1, PO-SPARC-1-1, (SEQ ID NOs: 7-8), were derived from si13347, si13346, and si13345 (SEQ ID NOs: 201-203), PO-SPARC-1-1 (SEQ ID NO: 8) and PO-SPARC-1 (SEQ ID NO:7) showed similar activity to si13347, si13346, and si13345 (SEQ ID NOs: 201-203), with exemplary results provided at FIGS. 5A and 5B. These results show that nucleotides complementary to BIO1 SPARC, including siRNAs and antisense oligonucleotides, are capable of inhibiting BIO1 in vitro.

### Example 3

This example demonstrates the measurement of cytotoxic activity of antisense oligonucleotides.

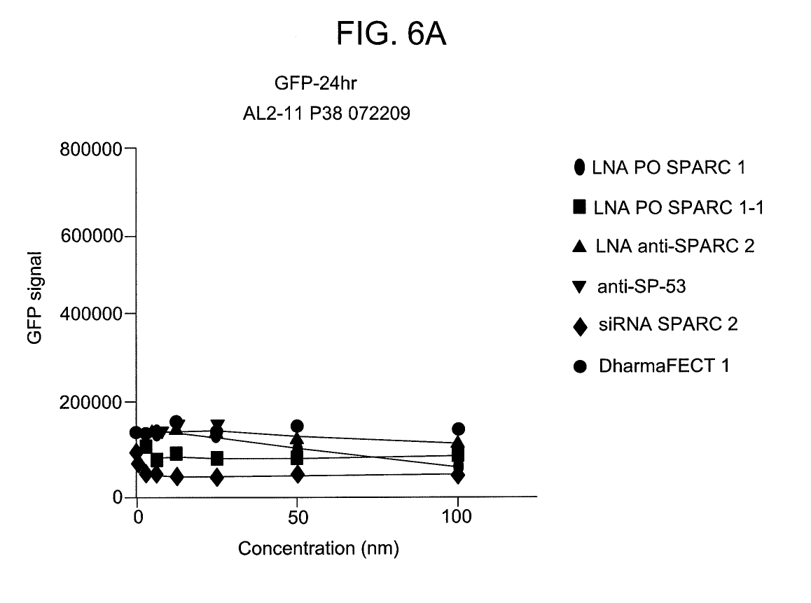

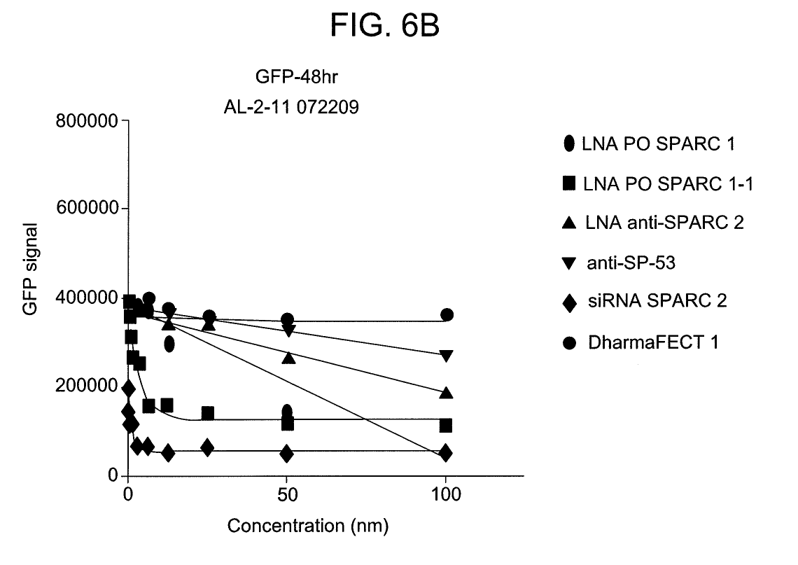

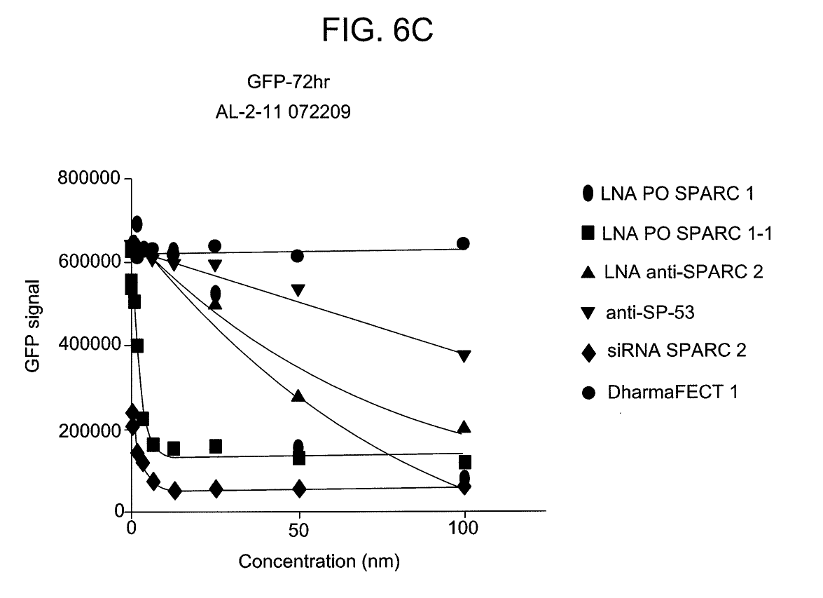

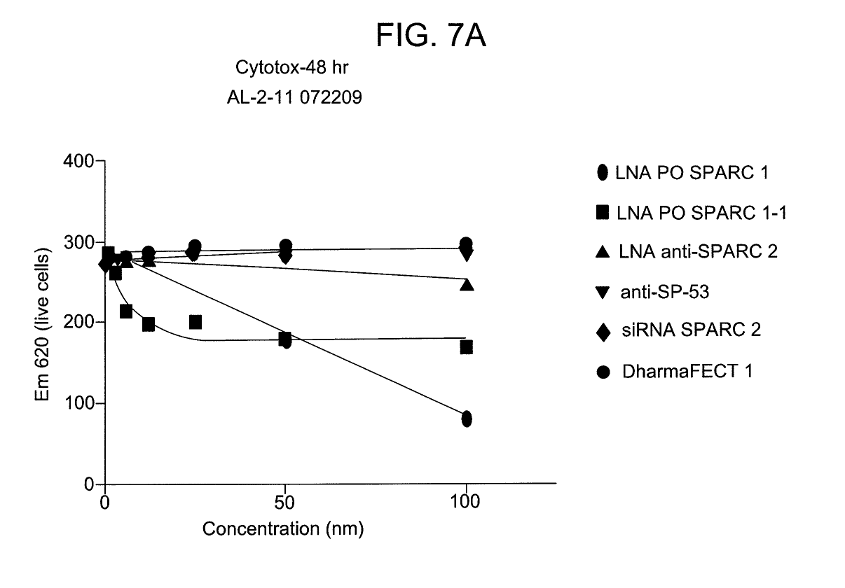

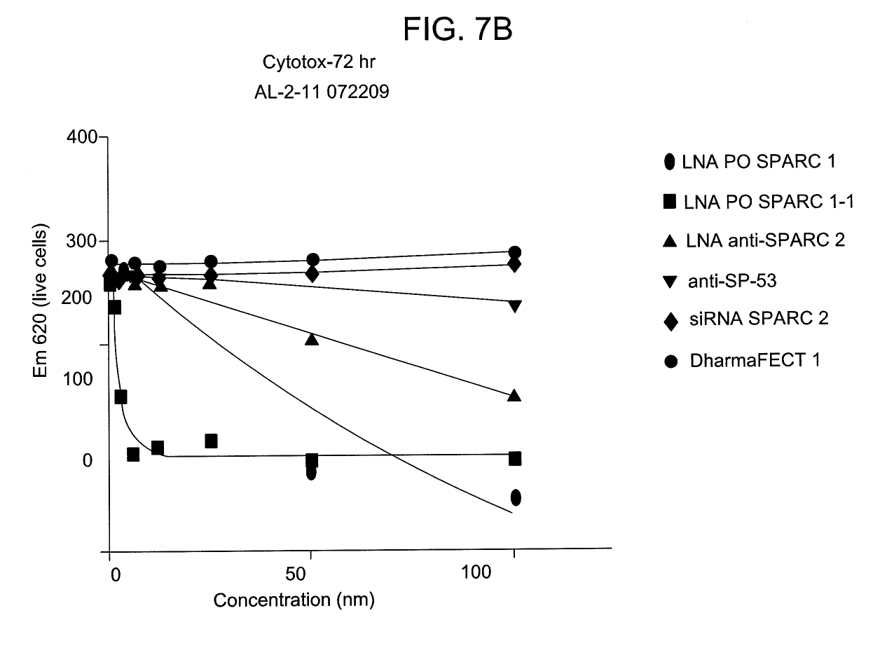

BIO1-GFP assays of nucleotides such as PO-SPARC1 (SEQ ID NO:7), PO-SPARC-1-1 (SEQ ID NO: 8), LNA anti-SPARC-2 (SEQ ID NO:3), anti-SP-53 (SEQ ID NO:66), siRNA-SPARC-2 (SEQ ID 202), and a negative control (DharmaFect1™ transfection agent (Dharmacon, Lafayette, Colo.)), were executed at concentrations up to 100 nM at 24, 48, and 72 hours as described in Example 2. Additionally, cytotoxicity was assayed at 48, and 72 hours by spectroscopy at 620 nm. In the GFP assays of Example 2, anti-SP-53 (SEQ ID NO: 66) exhibited minimal knock-down activity compared to the siRNAs, such as siRNA-SPARC-2 (FIGS. 6A-6C). However, anti-SP-53 exhibited notable cytotoxicity (FIGS. 7A-7B). LNA PO SPARC1, LNA-PO-SPARC-1-1, LNA anti-SPARC-2 showed both knock-down (FIG. 6A-C) and cytotoxicity (FIGS. 7A-B), while siRNA SPARC 2 had cytotoxic activity similar to the negative control.

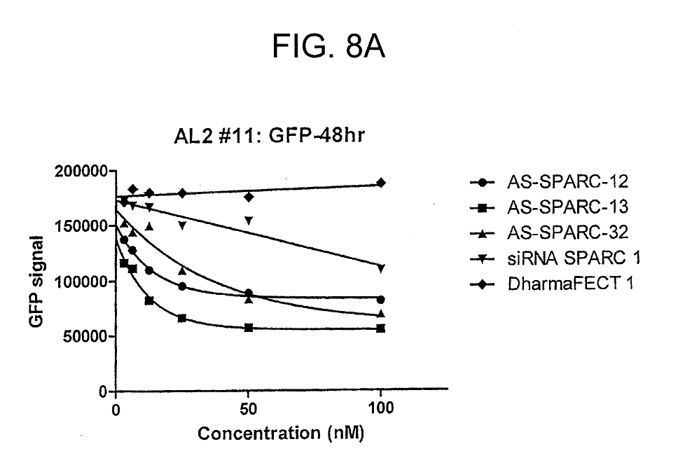

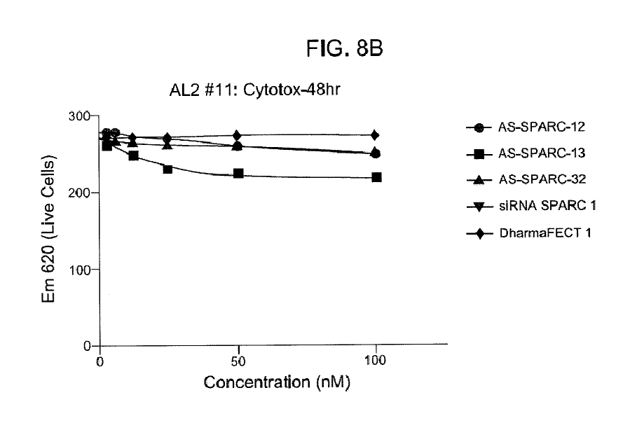

AS-SPARC-12 (SEQ ID NO:11), AS-SPARC-13 (SEQ ID NO:12), AS-SPARC-32 (SEQ ID NO:13), siRNA-SPARC-2 (SEQ ID NO:202) and a negative control (DharmaFect1™ transfection agent (Dharmacon, Lafayette, Colo.)), were similarly assayed for knock-down activity and cytotoxicity at 48 hours. AS-SPARC-12, AS-SPARC-13, AS-SPARC-32 each showed both strong knock-down activity and cytotoxic activity (FIG. 8A-8B). In contrast, siRNA-SPARC-2 showed knock-down activity without cytotoxic activity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A SPARC antisense composition comprising one or more of a DNA, RNA, LNA or PNA oligonucleotides comprising a nucleic acid sequence which is at least 90% identical to the sequence of any one of SEQ ID NOS: 2-4, 7-8, and 11-13, wherein the administration of the SPARC antisense composition to a cell reduces the level of SPARC protein in that cell by at least 30%.

3. The SPARC antisense composition of claim 2, wherein the one or more DNA, RNA, LNA or PNA oligonucleotide comprises any one or more of SEQ ID NOS: 11, 12, and 13.

4. The SPARC antisense composition of claim 2, wherein the DNA, RNA, LNA or PNA oligonucleotide comprises the nucleic acid sequence of SEQ ID NO: 11.

5. The SPARC antisense composition of claim 2, wherein the DNA, RNA, LNA or PNA oligonucleotide comprises the nucleic acid sequence of SEQ ID NO: 12.

6. The SPARC antisense composition of claim 2, wherein the DNA, RNA, LNA or PNA oligonucleotide comprises the nucleic acid sequence of SEQ ID NO: 13.

7. The SPARC antisense composition of claim 2, wherein the one or more DNA, RNA, LNA or PNA oligonucleotides comprises the nucleic acid sequence of any one or more of SEQ ID NOS: 2, 3, and 4.

8. The SPARC antisense composition of claim 2, wherein the DNA, RNA, LNA or PNA oligonucleotide comprises the nucleic acid sequence of SEQ ID NO: 2.

9. The SPARC antisense composition of claim 2, wherein the DNA, RNA, LNA or PNA oligonucleotide comprises the nucleic acid sequence of SEQ ID NO: 3.

10. The SPARC antisense composition of claim 2, wherein sa the id DNA, RNA, LNA or PNA oligonucleotide comprises the nucleic acid sequence of SEQ ID NO: 4.

11. The SPARC antisense composition of claim 2, wherein the one or more DNA, RNA, LNA or PNA oligonucleotides comprises the nucleic acid sequence of any one or more of SEQ ID NOS: 7 and 8.

12. The SPARC antisense composition of claim 2, wherein the DNA, RNA, LNA or PNA oligonucleotide comprises the nucleic acid sequence of SEQ ID NO: 7.

13. The SPARC antisense composition of claim 2, wherein the DNA, RNA, LNA or PNA oligonucleotide comprises the nucleic acid sequence of SEQ ID NO: 8.

14. (canceled)

15. The SPARC antisense composition of claim 2, wherein the oligonucleotide comprises a gapmer, mixmer, 2′-MOE, phosphorothioate boranophosphate, 2′-O-methyl, T-fluoro, terminal inverted-dT bases, PEG, 2′ tBDMS, 2′-TOM, f-ACE or combinations thereof.

16. The SPARC antisense composition of claim 2, wherein at least one of the one or more DNA, RNA, LNA or PNA oligonucleotides is modified by the addition of any one of cholesterol, bis-cholesterol, PEG, PEG-ylated carbon nanotube, poly-L-lysine, cyclodextran, polyethylenimine polymer, peptide moieties or a cell penetrating peptides.

17. The SPARC antisense composition of claim 2, wherein each of the one or more DNA, RNA, LNA or PNA oligonucleotides is modified by the addition of any one of cholesterol, bis-cholesterol, PEG, PEG-ylated carbon nanotube, poly-L-lysine, cyclodextran, polyethylenimine polymer, peptide moieties or a cell penetrating peptide.

18. The SPARC antisense composition of claim 2, further comprising a pharmaceutically-acceptable carrier.

19. A method of treating or preventing a proliferative disease in an animal comprising administering a therapeutically effective amount of the SPARC antisense composition of claim 2.

20. The method of claim 19, further comprising administering one or more therapeutic agents selected from the group consisting of chemotherapeutic agents, antiangiogenic agents, and kinase inhibitors.

21. The method of claim 20, wherein the chemotherapeutic agent is selected from the group consisting of ppactlitaxel, docetaxel, sutent, avastin, 5FU, adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin and its derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, combretastatins, discodermolides, and transplatinum.

22. The method of claim 19, wherein the SPARC antisense composition is administered directly to a diseased tissue in the animal, intravenously, subcutaneously, intramuscularly, nasally, intraperitonealy, vagainally, anally, orally, intraocularly or intrathecally.

23. The method of claim 19, wherein the SPARC antisense composition is administered to the animal for treatment or prevention of cancer, restenosis or other proliferative diseases, fibrosis, osteoporosis or exaggerated wound healing.

24. The method of claim 23, wherein
(a) the cancer is selected from the group consisting of circinoma in situ, atypical hyperplasia, carcinoma, sarcoma, carcinosarcoma, lung cancer, pancreatic cancer, skin cancer, melanoma, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof,
(c) the other proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osier-Weber syndrome, psoriasis, pyogenic granuloma, scleroderma, tracoma, menorrhagia, vascular adhesions, and papillomas, and
(d) the fibrotic disease disease is selected from the group consisting of hepatic fibrosis, pulmonary fibrosis and retroperitoneal fibrosis.

25. The method of claim 19, wherein the animal is undergoing one or more cancer therapies selected from the group consisting of surgery, chemotherapy, radiotherapy, thermotherapy, immunotherapy, hormone therapy and laser therapy.

26. A method of treating a proliferative disease in an animal comprising:
(a) isolating RNA from lesional tissue in the animal,
(b) isolating RNA from corresponding normal tissue,
(c) measuring the level of SPARC RNA the lesional tissue,
(d) measuring the level of SPARC RNA in the corresponding normal tissue,
(e) comparing the level of SPARC RNA in the lesional tissue with the level of SPARC RNA in the corresponding normal tissue, and
(f) administering a therapeutically effective amount of an SPARC antisense composition of claim 2 to the animal when the comparison in step (e) indicates that there exists a higher level of SPARC RNA in the lesional tissue relative to the level of SPARC RNA in the corresponding normal tissue.

27. The method of treating a proliferative disease of claim 24, wherein the level of SPARC RNA is determined by in situ hybridization, blot hybridization, PCR, TMA, invader or microarray.

28. A method of treating a proliferative disease in an animal comprising:
(a) isolating protein from lesional tissue in the animal,
(b) isolating protein from corresponding normal tissue,
(c) measuring the level of SPARC protein said lesional tissue,
(d) measuring the level of SPARC protein in said corresponding normal tissue,(e) comparing the level of SPARC protein in said lesional tissue with the level of SPARC RNA in said corresponding normal tissue, and
(f) administering a therapeutically effective amount of an SPARC antisense composition of claim 2 to the animal when the comparison in step (e) indicates that there exists a higher level of SPARC protein in the lesional tissue relative to the level of SPARC protein in the corresponding normal tissue.

29. The method of treating a proliferative disease of claim 28, wherein the level of SPARC protein is determined by immunohistology, immunoblot, antibody microarray or mass spectroscopy.

30. The method of claim 19, wherein the SPARC antisense composition is administered directly to the diseased tissue in the organism, intravenously, subcutaneously, intramuscularly, nasally, intraperitonealy, vagainally, anally, orally, intraocularly or intrathecally.

31. The method of claim 19, wherein the SPARC antisense composition is administered to the animal for treatment or prevention of cancer, restenosis or other proliferative diseases, fibrosis, osteoporosis or exaggerated wound healing.

32. The method of claim 31, wherein
(a) the cancer is selected from the group consisting of circinoma in situ, atypical hyperplasia, carcinoma, sarcoma, carcinosarcoma, lung cancer, pancreatic cancer, skin cancer, melanoma, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof,
(c) the other proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osier-Weber syndrome, psoriasis, pyogenic granuloma, scleroderma, tracoma, menorrhagia, vascular adhesions, and papillomas, and
(d) the fibrotic disease disease is selected from the group consisting of hepatic fibrosis, pulmonary fibrosis and retroperitoneal fibrosis.

33. The method of claim 19, wherein the animal is also undergoing one or more cancer therapies selected from the group consisting of surgery, chemotherapy, radiotherapy, thermotherapy, immunotherapy, hormone therapy and laser therapy.

34. The method of claim 19, wherein the animal is a human patient.

35. A SPARC antisense composition comprising one or more of a DNA, RNA, LNA or PNA oligonucleotide having a nucleic acid sequence complementary to SEQ ID NO: 1 at one or more of nucleotides 212, 311, 312, 521, 825, 841, 969, 985, 1001, 1017 of SEQ ID NO: 1, wherein the oligonucleotide is 12 to 19 nucleotides in length.
